(19) United States
(12) Patent Application Publication
(10) Pub. No.: US 2022/0251477 A1
(43) Pub. Date: Aug. 11, 2022

(54) CLEANING AGENTS INCLUDING ENZYME

(71) Applicant: Henkel AG & Co. KGaA, Duesseldorf (DE)

(72) Inventors: Thomas WEBER, Weimar (Lahn) (DE); Nicole BODE, Duesseldorf (DE); Thomas DOERING, Dormagen (DE); Klaus DORRA, Duesseldorf (DE); Britta STRAUSS, Solingen (DE); Nina MUSSMANN, Willich (DE); Christian DEGERING, Erkrath (DE); Thorsten BASTIGKEIT, Wuppertal (DE); Susanne WIELAND, Zons/Dormagen (DE)

(21) Appl. No.: 17/628,908

(22) PCT Filed: Jul. 16, 2020
(86) PCT No.: PCT/EP2020/070133

§ 371 Date: Jan. 21, 2022

(30) Foreign Application Priority Data
Jul. 22, 2019 (EP) 19187523.6

(51) Int. Cl. C11D 3/386; C11D 17/00; C11D 17/04

(52) U.S. Cl. CPC C11D 3/386; C11D 17/045; C11D 17/003

(57) ABSTRACT

Cleaning agents, such as dishwasher detergents, in particular dishwashing machine detergents, may include at least one gel-like phase having a low content of water, such as a substantially anhydrous gel-like phase having at least one enzyme, such as at least one enzyme granulate.

### REFERENCE TO A SEQUENCE LISTING SUBMITTED VIA EFS-WEB

The content of the ASCII text file of the sequence listing named 2019P00253WOUS_Sequence_Listing.txt, which is 22 kb in size was created on Jul. 22, 2019 and electronically submitted via EFS-Web herewith; the sequence listing is incorporated by reference in its entirety.

### CROSS-REFERENCE TO RELATED APPLICATIONS

The present application is a national stage entry according to 35 U.S.C. § 371 of PCT Application No. PCT/EP2020/070133 filed on Jul. 16, 2020; which claims priority to European Patent Application Serial No. 19187523.6 filed on Jul. 22, 2019; all of which are incorporated herein by reference in their entirety and for all purposes.

The disclosure relates to a cleaning agent, preferably a dishwasher detergent, in particular an automatic dishwasher detergent, comprising at least one low-water, preferably substantially water-free, gel phase which contains at least one enzyme, preferably at least one enzyme granulate.

Washing or cleaning agents are usually present in solid form (as tablets, for example) or in liquid form (or also as a flowing gel). Liquid washing or cleaning agents in particular are increasingly popular with consumers. Pre-portioned forms are popular with consumers because they are easier to meter. Pre-portioned flowing gels are often problematic because they tend to leak, for example when packed in single or multi-chamber bags.

The problem addressed is that of providing cleaning agents, in particular dishwasher detergents, preferably automatic dishwasher detergents, which can be produced easily and cost-effectively, while at the same time improving the cleaning performance, in particular on enzyme-sensitive stains.

A cleaning agent may include at least one low-water, preferably substantially water-free, gel phase which contains at least one enzyme.

An advantage is that the resulting cleaning agents have good stability, in particular stability during storage, as well as improved cleaning performance, in particular with respect to stains such as crème brûlée, egg yolk and spaghetti.

Surprisingly, it has been found that the cleaning agents have a particularly good cleaning performance on enzyme-sensitive stains. It is particularly noteworthy that the cleaning agents also have an improved cleaning performance on non-protease-specific stains.

In a preferred embodiment, the cleaning agent, preferably dishwasher detergent, in particular automatic dishwasher detergent, comprises at least one low-water, preferably substantially water-free, gel phase which contains at least one enzyme granulate. The at least one enzyme is therefore preferably in the form of a granulate. Particularly preferably, all the enzymes in the gel phase are in the form of a granulate.

This has the advantage that the stability of the enzyme or enzymes is very well guaranteed. In addition, visible particles, for example from enzyme granules, in transparent cleaning agent phases are very popular with consumers due to their attractive appearance.

The enzyme(s) contained in the gel phase include, in particular, proteases, amylases, lipases, hemicelluloses, cellulases, perhydrolases, or oxidoreductases, as well as, preferably, mixtures thereof. These enzymes are in principle of natural origin; starting from the natural molecules, variants that have been improved for use in cleaning agents are available, which are preferably used accordingly. Cleaning agents preferably contain enzymes in total quantities of from 1×10⁻⁶ wt. % to 5 wt. % based on active protein. The protein concentration can be determined using known methods, for example the BCA method or the Biuret method.

According to a preferred embodiment, the proportion by weight of all enzymes (based on the amount of active enzyme protein) in the at least one gel phase, based on the total weight of the gel phase(s), is 0.001 to 10.0 wt. %, preferably 0.01 to 7.5 wt. %, more preferably 0.05 to 6.0 wt. % and even more preferably 0.1 to 5.0 wt. %.

Among the proteases, the subtilisin-type proteases are preferred. Examples of these are the subtilisins BPN′ and Carlsberg, as well as the developed forms thereof, protease PB92, subtilisins 147 and 309, the alkaline protease from Bacillus lentus, subtilisin DY, and the enzymes thermitase, proteinase K and proteases TW3 and TW7, which belong to the subtilases but no longer to the subtilisins in the narrower sense.

According to a particularly preferred embodiment, the at least one gel phase comprises at least one enzyme, preferably at least one enzyme granulate, selected from the group of proteases, preferably selected from the group of subtilisins (or a subtilisin-containing enzyme), wherein the preferred protease(s) is/are selected from the following proteases:
a) protease which comprises an amino acid sequence which has at least 70% sequence identity with the amino acid sequence specified in SEQ ID NO:1 (B. gibsonii wt) over the entire length thereof and has an amino acid substitution at at least one of the positions corresponding to positions 12, 43, 122, 127, 154, 156, 160, 211, 212 or 222, each based on the numbering according to SEQ ID NO:1, in particular the at least one amino acid substitution being selected from the group consisting of Q12L, I43V, M122L, D127P, N154S, T156A, G160S, M211N, M21 IL, P212D, P212H or A222S, in each case based on the numbering according to SEQ ID NO:1, and/or particularly preferably having one of the following amino acid substitution variants, in each case based on the numbering according to SEQ ID NO:1:
   (i) I43V;
   (ii) M122L, N154S and T156A;
   (iii) M211N and P212D;
   (iv) M211L and P212D;
   (v) G160S;
   (vi) D127P, M211L and P212D;
   (vii) P212H; or
   (viii) Q12L, M122L and A222S;
b) protease which comprises an amino acid sequence which comprises a subtilisin 309 from Bacillus lentus, in particular one which comprises an amino acid sequence which is at least 80%, preferably at least 90%, in particular 100%, identical to the amino acid sequence specified in SEQ ID NO:2 over the entire length thereof and has at least one amino acid substitution at one of positions 9, 15, 66, 212 and 239 in the numbering according to SEQ ID NO:2, preferably selected from the group consisting of S9R, A15T, V66A, N212D and Q239R;
c) protease which comprises an amino acid sequence which comprises a subtilisin 309 from Bacillus lentus, in particular one which comprises an amino acid sequence which is at least 80%, preferably at least 90%, in particular 100%, identical to the amino acid sequence specified in SEQ ID NO:2 over the entire length thereof and has an amino acid substitution at position 97 and an insertion of an amino acid between the amino acids at positions 97 and 98 in the numbering according to SEQ ID NO:2, preferably selected from S97A and/or S97AD;
d) protease which comprises an amino acid sequence which has an amino acid sequence which is at least 80%, preferably at least 90%, in particular 100%, identical to the amino acid sequence specified in SEQ ID NO:3 (Bacillus alkalophilus PB92) over the entire length thereof and optionally has at least one amino acid substitution at one of the following positions 32, 33, 48-54, 58-62, 94-107, 116, 123-133, 150, 152-156, 158-161, 164, 169, 175-186, 197, 198 and 203-216 in the numbering according to SEQ ID NO:3, in particular at least one amino acid substitution at one, two, three or four of the following positions 116, 126, 127, 128 and 160 in the numbering according to SEQ ID NO:3, preferably at least one, even more preferably a plurality, most preferably each, of the amino acid substitutions G116V, S126L, P127Q and/or S128A and/or
e) protease which comprises an amino acid sequence which comprises an alkaline protease from Bacillus lentus DSM 5483, in particular one which has an amino acid sequence which is at least 80%, preferably at least 90%, in particular 100%, identical to the amino acid sequence specified in SEQ ID NO:4 over the entire length thereof and optionally has at least one amino acid substitution at one, two, three or four of the following positions 3, 4, 99 and 199 in the numbering according to SEQ ID NO:4, in particular the amino acid substitution R99E or R99D, and optionally additionally at least one or two, preferably all three, of the amino acid substitutions S3T, V4I and V199I.

In the context, the feature whereby an enzyme, in particular a protease, has the stated substitutions means that it contains at least one of the corresponding amino acids at the corresponding positions, i.e., not all of the 10 positions are otherwise mutated or deleted, for example by fragmentation of the enzyme, in particular the protease.

“Variant,” as used herein, relates to naturally or artificially generated variations of a native enzyme which have an amino acid sequence which is modified from the reference form. In addition to the amino acid alterations discussed above, enzymes can have other amino acid alterations, in particular amino acid substitutions, insertions or deletions. Such enzymes are, for example, developed by targeted genetic alteration, i.e., by mutagenesis methods, and optimized for specific applications or with regard to specific properties (e.g. with regard to their catalytic activity, stability, etc.). Furthermore, nucleic acids can be introduced into recombination approaches and can thus be used to generate completely new types of enzymes or other polypeptides. The aim is to introduce targeted mutations such as substitutions, insertions or deletions into the known molecules in order, for example, to improve the cleaning performance of enzymes. For this purpose, in particular the surface charges and/or the isoelectric point of the molecules and thus their interactions with the substrate can be altered. For instance, the net charge of the enzymes can be altered in order to influence the substrate binding, in particular for use in washing and cleaning agents. Alternatively or additionally, one or more corresponding mutations can increase the stability or catalytic activity of the enzyme and thus improve its cleaning performance. Advantageous properties of individual mutations, e.g., individual substitutions, can complement one another. A protease that has already been optimized with regard to certain properties can therefore be further developed, for example with regard to its stability towards surfactants and/or bleaching agents and/or other components.

For the description of substitutions relating to exactly one amino acid position (amino acid exchanges), the following convention is used herein: first, the naturally occurring amino acid is designated in the form of the internationally used one-letter codes, followed by the associated sequence position and finally the inserted amino acid. Several exchanges within the same polypeptide chain are separated by slashes. For insertions, additional amino acids are named following the sequence position. In the case of deletions, the missing amino acid is replaced by a symbol, for example a star or a dash, or a Δ is given before the corresponding position. For example, P14H describes the substitution of proline at position 14 by histidine, P14HT the insertion of threonine after the amino acid histidine at position 14 and P14* or ΔP14 the deletion of proline at position 14. This nomenclature is known to a person skilled in the field of enzyme technology.

The identity of nucleic acid or amino acid sequences is determined by a sequence comparison. This sequence comparison is based on the BLAST algorithm established and commonly used in the prior art (cf. e.g., Altschul et al. (1990) “Basic local alignment search tool,” J. Mol. Biol. 215:403-410, and Altschul et al. (1997): “Gapped BLAST and PSI-BLAST: a new generation of protein database search programs,” Nucleic Acids Res., 25:3389-3402) and occurs in principle by similar sequences of nucleotides or amino acids in the nucleic acid or amino acid sequences being associated with one another. A tabular association of the positions concerned is referred to as alignment. Another algorithm available in the prior art is the FASTA algorithm. Sequence comparisons (alignments), in particular multiple sequence comparisons, are created using computer programs. The Clustal series (cf. e.g., Chenna et al. (2003) “Multiple sequence alignment with the Clustal series of programs,” Nucleic Acid Res. 31: 3497-3500), T-Coffee (c.f. e.g., Notredame et al. (2000) “T-Coffee: A novel method for multiple sequence alignments,” J. Mol. Biol. 302:205-217) or programs based on these programs or algorithms, for example, are frequently used. Sequence comparisons (alignments) using the computer program Vector NTI® Suite 10.3 (Invitrogen Corporation, 1600 Faraday Avenue, Carlsbad, Calif., USA) with the predetermined standard parameters, the AlignX module of which program for the sequence comparisons is based on ClustalW, are also possible. Unless stated otherwise, the sequence identity specified herein is determined by the BLAST algorithm.

In a further embodiment, the protease used comprises an amino acid sequence which, over the entire length thereof, is preferably at least 70%, and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% and 98.8% identical to the amino acid sequence specified in SEQ ID NO:1, and preferably has, at at least one of the positions corresponding to positions 12, 43, 122, 127, 154, 156, 160, 211, 212 or 222 in the numbering according to SEQ ID NO:1, one or more of the amino acid substitutions 12L, 43V, 122L, 127P, 154S, 156A, 160S, 211N, 211L, 212D, 212H or 222S.

In a further embodiment, the protease used comprises an amino acid sequence which, over the entire length thereof, is at least 81% and increasingly preferably at least 82%, 83%, 84%, 85%, 86%, 87%, 88%, 89%, 90%, 9.5%, 91%, 91.5%, 92%, 92.5%, 93%, 93.5%, 94%, 94.5%, 95%, 95.5%, 96%, 96.5%, 97%, 97.5%, 98%, 98.5% and 98.8% identical to the amino acid sequence specified in SEQ ID NO:2, and preferably has at least one amino acid substitution at one of the positions corresponding to positions 9, 15, 66, 212 and 239 in the numbering according to SEQ ID NO:2, and has one or more amino acid substitutions, preferably selected from the group consisting of 9R, 15T, 66A, 212D or 239R.

In a further embodiment, the protease used comprises an amino acid sequence which, over the entire length thereof, is at least 81% and increasingly preferably at least 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% and 98.8% identical to the amino acid sequence specified in SEQ ID NO:2, and has an amino acid substitution at position 97 and an insertion of an amino acid between the amino acids at positions 97 and 98 in the numbering according to SEQ ID NO:2, preferably selected from 97A and/or 97AD.

In a further embodiment, the protease used comprises an amino acid sequence which, over the entire length thereof, is at least 81% and increasingly preferably at least 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% and 98.8% identical to the amino acid sequence specified in SEQ ID NO:3, and preferably, at at least one of the positions corresponding to positions 32, 33, 48-54, 58-62, 94-107, 116, 123-133, 150, 152-156, 158-161, 164, 169, 175-186, 197, 198 and 203-216 in the numbering according to SEQ ID NO:3, has in particular at least one amino acid substitution at one, two, three or four of the following positions 116, 126, 127, 128 and 160 in the numbering according to SEQ ID NO:3, preferably at least one, more preferably a plurality of, most preferably each of amino acid substitutions 116V, 126L, 127Q, and/or 128A.

In a further embodiment, the protease used comprises an amino acid sequence which, over the entire length thereof, is at least 81% and increasingly preferably at least 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% and 98.8% identical to the amino acid sequence specified in SEQ ID NO:4, and preferably, at at least one of the positions corresponding to positions 3, 4, 99 and 199 in the numbering according to SEQ ID NO:4, has in particular the amino acid substitution 99E or 99D, and optionally additionally at least one or two, preferably all three, of the amino acid substitutions 3T, 4I and 199I.

According to a further particularly preferred embodiment, the amount of all proteases, based on the amount of active enzyme protein which is added per wash cycle, is preferably 0.001 to 1000 mg/job, more preferably 0.1 to 600 mg/job and even more preferably 1.0 to 400 mg/job.

Particularly preferably, the at least one gel phase comprises as an enzyme (optionally in addition to further enzymes) a protease which comprises an amino acid sequence which has at least 70% sequence identity with the amino acid sequence specified in SEQ ID NO:1 (B. gibsonii wt) over the entire length thereof and has an amino acid substitution at at least one of the positions corresponding to positions 12, 43, 122, 127, 154, 156, 160, 211, 212 or 222, each based on the numbering according to SEQ ID NO:1, in particular the at least one amino acid substitution being selected from the group consisting of Q12L, I43V, M122L, D127P, N154S, T156A, G160S, M211N, M211L, P212D, P212H or A222S, in each case based on the numbering according to SEQ ID NO:1, and/or particularly preferably having one of the following amino acid substitution variants, in each case based on the numbering according to SEQ ID NO:1:
(i) I43V;
(ii) M122L, N154S and T156A;
(iii) M211N and P212D;
(iv) M211L and P212D;
(v) G160S;
(vi) D127P, M211L and P212D;
(vii) P212H; or
(viii) Q12L, M122L and A222S.

The cleaning agent very particularly preferably contains 0.001 to 1000 mg, more preferably 0.1 to 600 mg and even more preferably 1.0 to 400 mg per cleaning agent portion, preferably in the gel phase of the cleaning agent, of a protease which comprises an amino acid sequence which has at least 70% sequence identity, over the entire length thereof, to the amino acid sequence specified in SEQ ID NO:1 (B. gibsonii wt), and has an amino acid substitution at at least one of the positions corresponding to positions 12, 43, 122, 127, 154, 156, 160, 211, 212 or 222, in each case based on the numbering according to SEQ ID NO:1, in particular the at least one amino acid substitution from the group consisting of Q12L, I43V, M122L, D127P, N154S, T156A, G160S, M211N, M211L, P212D, P212H or A222S, in each case based on the numbering according to SEQ ID NO:1, being selected, and/or particularly preferably having one of the following amino acid substitution variants, in each case based on the numbering according to SEQ ID NO:1:
(i) I43V;
(ii) M122L, N154S and T156A;
(iii) M211N and P212D;
(iv) M211L and P212D;
(v) G160S;
(vi) D127P, M211L and P212D;
(vii) P212H; or
(viii) Q12L, M122L and A222S.

According to another objective, it is also possible that in a cleaning agent, in particular in a single cleaning agent portion which comprises at least one gel phase and at least one further, preferably solid, phase comprising granular mixture, in particular powder, a protease from the group of proteases mentioned directly above is not incorporated into the gel phase, but rather can be partly or completely incorporated into the at least one further, preferably at least one solid phase, particularly preferably at least one further solid phase comprising a granular mixture. The amounts to be used based on the cleaning agent portion to be used remain unchanged. The embodiments described above or below for the gel phase and the further, preferably solid, phase also apply analogously to this embodiment.

According to a further preferred embodiment, the proportion by weight of all proteases, based on the amount of active enzyme protein of all proteases, with respect to the total weight of the cleaning agent is 0.005 to 5 wt. %, preferably 0.01 to 2.0 wt. %, more preferably 0.05 to 1.5 wt. %, and even more preferably 0.01 to 1.0 wt. %.

According to a further preferred embodiment, the proportion by weight of all proteases, based on the amount of active enzyme protein, in the at least one gel phase, based on the total weight of the gel phase(s), is 0.0005 to 20.0 wt. %, preferably 0.001 to 15 wt. %, more preferably 0.01 to 10.0 wt. % and even more preferably 0.1 to 7.5 wt. %.

According to a further preferred embodiment, the amount of all proteases, based on the amount of active enzyme protein which is added per wash cycle, is preferably 0.001 to 1000 mg/job, more preferably 0.1 to 600 mg/job and even more preferably 1.0 to 400 mg/job.

According to a further preferred embodiment, the at least one gel phase comprises at least one amylase, optionally in addition to other enzymes, in particular protease(s). Amylases which are preferred are α-amylases. Whether or not an enzyme is an α-amylase is decided by its ability to hydrolyze α-(1-4)-glycosidic bonds in polysaccharides, in particular amylose and starch. As a result, they cause starchy stains to break down on the items to be cleaned. Dextrins and from them maltose, glucose and branched oligosaccharides are formed as breakdown products.

Examples of amylases are α-amylases from Bacillus lichenformis, from Bacillus amyloliquefaciens or from Bacillus stearothermophilus, as well as in particular the developments thereof that have been improved for use in washing or cleaning agents. The enzyme from Bacillus lichenformis is available from Novozymes under the name Termamyl® and from Danisco/Genencor under the name Purastar® ST. Development products of this α-amylase are available from Novozymes under the trade names Duramyl® and Termamyl® ultra, from Danisco/Genencor under the name Purastar® OxAm, and from Daiwa Seiko Inc. as Keistase®. The α-amylase from Bacillus amyloliquefaciens is marketed by Novozymes under the name BAN®, and derived variants from the α-amylase from Bacillus stearothermophilus are marketed under the names BSG® and Novamyl®, also by Novozymes. Furthermore, for this purpose the α-amylases from Bacillus sp. A 7-7 (DSM 12368) and the cyclodextrin glucanotransferase (CGTase) from Bacillus agaradherens (DSM 9948) should be emphasized. Furthermore, the amylolytic enzymes can be used which are disclosed in international patent applications WO2003002711, WO2003054177 and WO2007079938, the disclosure of which is therefore expressly referred to or the disclosure of which is therefore expressly included in the present patent application. Fusion products of all mentioned molecules can also be used. Furthermore, the developments of the α-amylase from Aspergillus niger and A. oryzae, available under the trade name Fungamyl® from Novozymes, are suitable. Other commercial products that can advantageously be used are, for example, Amylase-LT®, and Stainzyme® or Stainzyme Ultra® or Stainzyme Plus® also from Novozymes. Variants of these enzymes that can be obtained by point mutations can also be used.

The α-amylase used in the washing or cleaning agents is preferably selected from:
i. an α-amylase which comprises an amino acid sequence which is at least 80%, preferably at least 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% and 98.8%, identical to the amino acid sequence specified in SEQ ID NO:5 over the entire length thereof and optionally has at least one amino acid substitution at one of the positions 172, 202, 208, 255 and 261 in the numbering according to SEQ ID NO:5, preferably selected from the group consisting of M202L, M202V, M202S, M202T, M202I, M202Q, M202W, S255N, R172Q and combinations thereof; and/or
ii. an α-amylase which comprises an amino acid sequence which is at least 60%, preferably at least 61%, 62%, 63%, 64%, 65%, 66%, 67%, 68%, 69%, 70% 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% and 98.8, identical to the amino acid sequence specified in SEQ ID NO:6 over the entire length thereof and optionally has at least one amino acid substitution at one of the positions 9, 26, 30, 33, 82, 37, 106, 118, 128, 133, 149, 150, 160, 178, 182, 186, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 378, 383, 419, 421, 437, 441, 444, 445, 446, 447, 450, 458, 461, 471, 482 and 484 and/or a deletion at one of the positions 183 and 184 in the numbering according to SEQ ID NO:6, preferably at least one amino acid substitution at one of the positions 9, 26, 149, 182, 186, 202, 257, 295, 299, 323, 339, and 345, particularly preferably selected from the group consisting of R118K, D183*, G184*, N195F, R320K, R458K and combinations thereof, and/or
iii. an α-amylase which comprises an amino acid sequence which is at least 90% identical to the amino acid sequence specified in SEQ ID NO:7 over the entire length thereof and optionally has at least one substitution and/or deletion at one of the positions 93, 116, 118, 129, 133, 134, 140, 142, 146, 147, 149, 151, 152, 169, 174, 183, 184, 186, 189, 193, 195, 197, 198, 200, 203, 206, 210, 212, 213, 235, 243, 244, 260, 262, 284, 303, 304, 320, 338, 347, 359, 418, 431, 434, 439, 447, 458, 469, 476 and 477 in the numbering according to SEQ ID NO:7.

In various embodiments, the amylase comprises an amino acid sequence which is at least 80%, preferably at least 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5% and 98.8%, identical to the amino acid sequence specified in SEQ ID NO:5 over the entire length thereof and optionally has at least one amino acid substitution at one of the positions 172, 202, 208, 255 and 261 in the numbering according to SEQ ID NO:5, preferably selected from the group consisting of M202L, M202V, M202S, M202T, M202I, M202Q, M202W, S255N, R172Q and combinations thereof. Amylases are preferably used which have an amino acid substitution at two, preferably three, of the above-mentioned positions, in particular a substitution at position 202 selected from M202L, M202V, M202S, M202T, M202I, M202Q, M202W, a substitution at position 255, in particular S255N, and a substitution at position 172, in particular R172Q. The M202L and M202T mutants are very particularly preferred.

In various embodiments, the amylase comprises an amino acid sequence which is at least 60%, preferably at least 61%, 62%, 63%, 64%, 65%, 66%, 67%, 68%, 69%, 70% 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5% 94%, 94.5% 95%, 95.5% 96%, 96.5%, 97%, 97.5%, 98%, 98.5% and 98.8, identical to the amino acid sequence specified in SEQ ID NO:6 over the entire length thereof and optionally has at least one amino acid substitution at one of the positions 9, 26, 30, 33, 82, 37, 106, 118, 128, 133, 149, 150, 160, 178, 182, 186, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 378, 383, 419, 421, 437, 441, 444, 445, 446, 447, 450, 458, 461, 471, 482 and 484 and/or a deletion at one of the positions 183 and 184 in the numbering according to SEQ ID NO:6, preferably at least one amino acid substitution at one of the positions 9, 26, 149, 182, 186, 202, 257, 295, 299, 323, 339 and 345, particularly preferably selected from the group consisting of R118K, D183*, G184*, N195F, R320K, R458K and combinations thereof. In various preferred embodiments, the amylase in the numbering according to SEQ ID NO:6 has an amino acid substitutions at three or more of the positions 9, 26, 149, 182, 186, 202, 257, 295, 299, 323, 339 and 345 and optionally one or more, preferably all, of the substitutions and/or deletions at the positions: 118, 183, 184, 195, 320 and 458, particularly preferably R118K, D183*, G184*, N195F, R320K and/or R458K. In particularly preferred embodiments, the amylase in the numbering according to SEQ ID NO:6 has the following amino acid substitutions and/or deletions: M9L+M323T; M9L+M202L/T/V/I+M323T; M9L+N195F+M202L/T/V/I+M323T; M9L+R118K+D183*+G184*+R320K+M323T+R458K; M9L+R118K+D183*+G184*+M202L/T/V/I+R320K+M323T+R458K; M9L+G149A+G182T+G186A+M202L+T257I+Y295F+N299Y+M323T+A339S+E345R; M9L+G149A+G182T+G186A+M202I+T257I+Y295F+N299Y+M323T+A339S+E345R; M9L+R118K+G149A+G182T+D183*+G184*+G186A+M202L+T257I+Y295F+N299Y+R320K+M323T+A339S+E345R+R458K; M9L+R118K+G149A+G182T+D183*+G184*+G186A+N195F+M202L+T257I+Y295F+N299Y+R320K+M323T+A339S+E345R+R458K; M9L+R118K+G149A+G182T+D183*+G184*+G186A+M202I+T257I+Y295F+N299Y+R320K+M323T+A339S+E345R+R458K; M9L+R118K+D183*+D184*+N195F+M202L+R320K+M323T+R458K; M9L+R118K+D183*+D184*+N195F+M202T+R320K+M323T+R458K; M9L+R118K+D183*+D184*+N195F+M202I+R320K+M323T+R458K; M9L+R118K+D183*+D184*+N195F+M202V+R320K+M323T+R458K; M9L+R118K+N150H+D183*+D184*+N195F+M202L+V214T+R320K+M323T+R458K; or M9L+R118K+D183*+D184*+N195F+M202L+V214T+R320K+M323T+E345N+R458K.

A particularly preferred amylase is the variant which is commercially available under the trade name Stainzyme Plus™ (Novozymes A/S, Bagsvaerd, Denmark).

In various embodiments, the amylase comprises an amino acid sequence which is at least 90%, preferably 90.5%, 91%, 91.5%, 92%, 92.5%, 93%, 93.5%, 94%, 94.5%, 95%, 95.5%, 96%, 96.5%, 97%, 97.5%, 98%, 98.5% and 98.8% identical to the amino acid sequence specified in SEQ ID NO:7 over the entire length thereof and optionally has at least one substitution and/or deletion at one of the positions 93, 116, 118, 129, 133, 134, 140, 142, 146, 147, 149, 151, 152, 169, 174, 183, 184, 186, 189, 193, 195, 197, 198, 200, 203, 206, 210, 212, 213, 235, 243, 244, 260, 262, 284, 303, 304, 320, 338, 347, 359, 418, 431, 434, 439, 447, 458, 469, 476 and 477 in the numbering according to SEQ ID NO:7. Preferred amino acid substitutions in this regard include: E260A/D/C/Q/L/M/F/P/S/WA/G/H/I/K/N/R/T/Y, G304R/K/E/Q, W140Y/F, W189E/G/T, D134E, F262G/P, W284D/H/F/Y/R, W347H/F/Y, W439R/G, G476E/Q/R/K, G477E/Q/K/M/R, N195F/Y, N197F/L, Y198N, Y200F, Y203F, I206H/L/N/F/Y, H210Y, E212V/G, V213A, M116T, Q129L, G133E, E134Y, K142R, P146S, G147E, G149R, N151R, Y152H, Q169E, N174R, A186R, Y243F, S244Q, G303V, R320N, R3591, N418D and A447V.

In various preferred embodiments, cleaning agents can contain at least one second amylase and/or at least one second protease, the second amylase being different from the first amylase and being selected from the group mentioned above, and the second protease being different from the first protease.

If two amylases are contained, these can preferably be used in a weight ratio of from 50:1 to 1:50, preferably 30:1 to 1:10 (in each case based on the amount of active protein amylase 1 to amylase 2). Using a first amylase in a ratio of from 20:1 to 2:1, preferably 15:1 to 3:1, particularly preferably 12:1 to 5:1, for example 10:1, to a second amylase is in particular preferred.

According to a preferred embodiment, the proportion by weight of the at least one amylase, based on the amount of active enzyme protein of all amylases, with respect to the total weight of the cleaning agent is 0.0005 to 2 wt. %, preferably 0.001 to 1.5 wt. %, more preferably 0.005 to 1.0 wt. %, and even more preferably 0.001 to 0.8 wt. %.

Furthermore, lipases or cutinases can be used, in particular due to their triglyceride-cleaving activities, but also in order to produce peracids in situ from suitable precursors. These include, for example, the lipases that could originally be obtained from Humicola lanuginosa (Thermomyces lanuginosus) and those that have been further developed, particularly those with the amino acid exchange in positions D96LT213R and/or N233R, especially preferably all of the exchanges D96L, T213R, and N233R.

Moreover, enzymes can be used which can be grouped together under the term “hemicellulases.” These include, for example, mannanases, xanthan lyases, pectin lyases (=pectinases), pectinesterases, pectate lyases, xyloglucanases (=xylases), pullulanases, and β-glucanases.

In order to increase the bleaching effect, oxidoreductases such as oxidases, oxygenases, catalases, peroxidases such as halo-, chloro-, bromo-, lignin, glucose, or manganese peroxidases, dioxygenases or laccases (phenoloxidases, polyphenoloxidases) can be used. Advantageously, organic, particularly preferably aromatic compounds that interact with the enzymes are additionally added in order to potentiate the activity of the relevant oxidoreductases (enhancers) or, in the event of greatly differing redox potentials, to ensure the flow of electrons between the oxidizing enzymes and the contaminants (mediators). A protein and/or enzyme can be protected, especially during storage, against damage such as inactivation, denaturing, or decomposition caused for example by physical influences, oxidation or proteolytic cleavage. When the proteins and/or enzymes are obtained microbially, it is particularly preferable for proteolysis to be inhibited, particularly if the agents also contain proteases. Cleaning agents may contain stabilizers for this purpose; the provision of such agents constitutes a preferred embodiment.

Cleaning-active proteases and amylases are generally not made available in the form of the pure protein, but rather in the form of stabilized, storable and transportable preparations. These ready-made preparations include, for example, the solid preparations obtained through granulation, extrusion, or lyophilization or, particularly in the case of liquid or gel agents which are advantageously maximally concentrated, have a low water content, and/or are supplemented with stabilizers or other auxiliaries.

Alternatively, the enzymes can also be encapsulated for the at least one gel phase, preferably solid gel phase, for example by spray-drying or extrusion of the enzyme solution together with a preferably natural polymer or in the form of capsules, for example those in which the enzymes are enclosed in a set gel, or in those of the core-shell type in which an enzyme-containing core is coated with a water-, air-, and/or chemical-impermeable protective layer. Other active ingredients such as stabilizers, emulsifiers, pigments, bleaching agents, or dyes can additionally be applied in overlaid layers. Such capsules are applied using methods which are known per se, for example by shaking or rolling granulation or in fluidized bed processes. Advantageously, such granules are low in dust, for example due to the application of polymeric film-formers, and stable in storage due to the coating.

Moreover, it is possible to formulate two or more enzymes together such that a single granule exhibits a plurality of enzyme activities.

As is clear from the preceding remarks, the enzyme protein forms only a fraction of the total weight of conventional enzyme preparations. Protease and amylase preparations used contain between 1 and 40 wt. %, preferably between 2 and 30 wt. %, particularly preferably between 3 and 25 wt. % of the enzyme protein. In particular, those cleaning agents are preferred which contain, based on their total weight, 0.1 to 12 wt. %, preferably 0.2 to 10 wt. %, and in particular 0.5 to 8 wt. % of the respective enzyme preparations.

A gel-like phase, hereinafter also referred to as gel phase, is to be understood as meaning a composition/phase which has an internally structuring network. This internally structuring (spatial) network is formed by the dispersion of a solid but distributed substance with long or highly branched particles and/or gelling agents, in at least one liquid (the at least one liquid is liquid at 20° C.). Gel phases of this kind are thermoreversible.

This gel phase can be, for example, flowable or dimensionally stable. However, the gel phase is preferably dimensionally stable at room temperature. During preparation, the gelling agent, preferably xanthan gum, gelatin or polyvinyl alcohol and/or derivatives thereof, is brought into contact with a solvent, preferably an organic solvent, preferably one or more polyhydric alcohol(s). This enables a flowable mixture to be obtained that can be molded into shape as desired. After a certain period of time, a gel phase is obtained which remains in the given form, i.e. is dimensionally stable. This time period, the setting time, is preferably 15 minutes or less, more preferably 10 minutes or less, particularly preferably 5 minutes or less. In this case, the at least one gel phase gives way to pressure, but does not deform, and instead returns to its initial state after the pressure has ceased. The at least one gel phase is preferably elastic, in particular linear-elastic.

The at least one gel phase is preferably a shaped body. A shaped body is a single body that stabilizes itself in the shape imparted to it. This dimensionally stable body is formed from a molding compound (e.g., a composition) in such a way that this molding compound is deliberately brought into a predetermined shape, for example by pouring a liquid composition into a casting mold and then curing the liquid composition, for example as part of a sol-gel process.

Certain minimum requirements apply to formulations of the at least one gel phase. As already mentioned, the gel phase must solidify within as short a time as possible. Long solidification times would result in long production time and thus lead to high costs. Solidification time means the period of time during which the at least one gel phase changes from a flowable state to a dimensionally stable state which is non-flowable at room temperature during preparation. Room temperature is to be understood as a temperature of 20° C.

The at least one gel phase is preferably a solid gel phase. It is therefore sliceable. For example, it can be cut with a knife after it sets without being destroyed beyond the cut that is made.

The at least one gel phase is also preferably translucent or transparent, as a result of which a good visual impression is achieved. Preferably, the transmission of the gel phase (without dye) is in a range between 100% and 20%, between 100% and 30%, in particular between 100% and 40%. To measure the light transmittance (transmission), the patency was determined in % at 600 nm against water as a reference at 20° C. For this purpose, the mass was poured into the designated 11 mm round cuvettes and measured after a 12 h storage period at room temperature in a Lange LICO 300 color measuring system.

At least one gel phase is low-water. Low-water means that small quantities of water can be used to prepare the at least one gel phase. The proportion of water in the gel phase is in particular 20 wt. % or less, preferably 15 wt. % or less, particularly 12 wt. % or less, in particular between 10 and 5 wt. %. The quantities in wt. % refer to the total weight of the gel phase. This has the advantage that the small amounts of water in combination with PVOH can have a structure-forming or gel-forming effect.

According to a preferred embodiment, the at least one gel phase is substantially water-free. This means that the gel phase is preferably substantially free of water. “Substantially free” means in this case that the gel phase may contain small quantities of water. For example, this water can be introduced into the phase by means of a solvent or as water of crystallization or as a result of reactions of components of the phase with each other. However, only small quantities of water, and in particular no water, are preferably used as a solvent for preparing the gel phase. The proportion of water in the gel phase in this embodiment is 4.9 wt. % or less, 4 wt. % or less, preferably 2 wt. % or less, in particular 1 wt. % or less, particularly 0.5 wt. % or less, in particular 0.1 wt. % or 0.05 wt. % or less. The quantities in wt. % refer to the total weight of the gel phase in this case.

These and other aspects, features, and advantages will become apparent to a person skilled in the art through the study of the following detailed description and claims. Any feature from one aspect can be used in any other aspect. Furthermore, it will readily be understood that the examples contained herein are intended to describe and illustrate but not to limit the invention and that, in particular, the invention is not limited to these examples. Unless indicated otherwise, all percentages are indicated in terms of wt. %. Numerical ranges that are indicated in the format “from x to y” also include the stated values. If several preferred numerical ranges are indicated in this format, it is readily understood that all ranges that result from the combination of the various endpoints are also included.

“At least one,” as used herein, means 1 or more, i.e., 1, 2, 3, 4, 5, 6, 7, 8, 9 or more. In relation to an ingredient, the expression refers to the type of ingredient and not to the absolute number of molecules. “At least one bleach catalyst” therefore means at least one type of bleach catalyst, for example—that is, that one type of bleach catalyst or a mixture of a plurality of different bleach catalysts can be used. Together with weight specifications, the expression relates to all compounds of the type indicated that are contained in the composition/mixture, i.e., the composition does not contain any other compounds of this type beyond the indicated amount of the corresponding compounds.

When reference is made herein to molar masses, this information always refers to the number-average molar mass Mn, unless explicitly indicated otherwise. The number-average molar mass can, for example, be determined by gel permeation chromatography (GPC) according to DIN 55672-1:2007-08 with THE as the eluent. The number-average molar mass MW can also be determined by means of GPC, as described for Mn.

Unless explicitly indicated otherwise, all percentages that are cited in connection with the compositions described herein relate to wt. %, in each case with respect to the relevant mixture or phase.

Furthermore, the gel phase must be storage stable under normal storage conditions. The gel phase is a component of a cleaning agent. Cleaning agents are usually stored for a certain period of time in a household. They are usually stored near the washing machine or dishwasher. For such storage, the gel phase should be stable. Therefore, the gel phase should be stable, especially after a storage period of 4 to 12 weeks, especially 10 to 12 weeks or longer at a temperature of up to 40° C., particularly at 30° C., in particular at 25° C. or at 20° C., and should not deform or otherwise change in consistency during this time.

If the gel phase and a solid phase, in particular a powder phase, are present in direct contact with each other, the gel phase preferably penetrates a maximum of 1 mm into the interstices of the immediately underlying powder phase during the storage period of 4 weeks at 25° C.

A change in volume or shrinkage during storage would be disadvantageous, since that would diminish consumers' acceptance of the product. A leakage of liquid or components sweating out of the gel phase is also undesirable. Here, too, the visual impression is relevant, for one thing. The stability of the gel phase can be influenced by the leakage of liquid, such as solvents, such that the components are no longer stably contained and the washing or cleaning effect can also be influenced as a result.

According to a preferred embodiment, the cleaning agents, preferably dishwasher detergents, in particular automatic dishwasher detergents, contain in the gel phase a gelling agent, preferably selected from gelatin, xanthan gum and/or polyvinyl alcohol, in particular gelatin or polyvinyl alcohol, particularly preferably polyvinyl alcohol, in a quantity of from 4 to 40, in particular of from 6 to 30 wt. %, particularly preferably in a quantity of from 7 to 24 wt. %, more particularly preferably 8 to 22 wt. %, in particular for example 14 to 20 wt. %, in each case based on the total weight of the gel phase.

The at least one gel phase particularly preferably comprises PVOH (polyvinyl alcohol) and/or derivatives thereof. Polyvinyl alcohols are thermoplastic materials that are manufactured as white to yellowish powders, usually by hydrolysis of polyvinyl acetate. Polyvinyl alcohol (PVOH) is resistant to almost all water-free organic solvents. Polyvinyl alcohols having a molar mass of from 30,000 to 60,000 g/mol are preferred. Derivatives of PVOH are preferably copolymers of polyvinyl alcohol with other monomers, in particular copolymers with anionic monomers. Suitable anionic monomers are preferably vinyl acetic acid, alkyl acrylates, maleic acid and derivatives thereof, in particular monoalkyl maleates (in particular monomethyl maleate), dialkyl maleates (in particular dimethyl maleate), maleic anhydride, fumaric acid and derivatives thereof, in particular monoalkyl fumarate (in particular monomethyl fumarate), dialkyl fumarate (in particular dimethyl fumarate), fumaric anhydride, itaconic acid and derivatives thereof, in particular monomethyl itaconate, dialkyl itaconate, dimethyl itaconate, itaconic anhydride, citraconic acid (methylmaleic acid) and derivatives thereof, monoalkyl citraconic acid (in particular methyl citraconate), dialkyl citraconic acid (dimethyl citraconate), citraconic anhydride, mesaconic acid (methyl fumaric acid) and derivatives thereof, monoalkyl mesaconate, dialkyl mesaconate, mesaconic anhydride, glutaconic acid and derivatives thereof, monoalkyl glutaconate, dialkyl glutaconate, glutaconic anhydride, vinylsulfonic acid, alkyl sulfonic acid, ethylene sulfonic acid, 2-acrylamido-1-methylpropanesulfonic acid, 2-acrylamido-2-methylpropanesulfonic acid, 2-methylacrylamido-2-methylpropanesulfonic acid, 2-sulfoethyl acrylate and combinations thereof, and the alkali metal salts or esters of the above-mentioned monomers.

Particular preferred derivatives of PVOH are those selected from copolymers of polyvinyl alcohol with a monomer, in particular selected from the group of monoalkyl maleates (in particular monomethyl maleate), dialkyl maleates (in particular dimethyl maleate), maleic anhydride, and combinations thereof, and the alkali salts or esters of the above-mentioned monomers. The values stated for polyvinyl alcohols themselves apply to the suitable molecular masses. It is preferred for the at least one gel phase to comprise a polyvinyl alcohol and/or derivatives thereof, preferably polyvinyl alcohol, of which the degree of hydrolysis is preferably 70 to 100 mol. %, in particular 80 to 90 mol. %, particularly preferably 81 to 89 mol. %, and even more preferably 82 to 88 mol. %.

Particularly preferred polyvinyl alcohols are those present as white-yellowish powders or granulates having degrees of polymerization in the range of from approximately 100 to 2,500 (molar masses of from approximately 4,000 to 100,000 g/mol) and degrees of hydrolysis of from 80 to 99 mol. %, preferably from 85 to 90 mol. %, in particular from 87 to 89 mol. %, for example 88 mol. %, which accordingly also contain a residual content of acetyl groups.

PVOH powders which have the above-mentioned properties and are suitable for use in the at least one gel phase are marketed by Kuraray, for example, under the name Mowiol® or Poval®. Exceval® AQ4104 from Kuraray is also suitable, for example. Particularly suitable are Mowiol C30, the Poval® grades, in particular qualities 3-83, 3-88, 6-88, 4-85, and particularly preferably 4-88, very particularly preferably Poval 4-88 S2, and Mowiol® 4-88 from Kuraray.

The water solubility of polyvinyl alcohol can be altered by post-treatment with aldehydes (acetalization) or ketones (ketalization). Particularly preferred and, due to their decidedly good solubility in cold water, particularly advantageous polyvinyl alcohols have been produced which can be acetalized or ketalized with the aldehyde or keto groups of saccharides or polysaccharides or mixtures thereof. It is extremely advantageous to use the reaction products of polyvinyl alcohol and starch. Furthermore, the water solubility can be altered and thus set at desired values in a targeted manner by complexing with Ni or Cu salts or by treatment with dichromates, boric acid, or borax.

According to a preferred embodiment, the at least enzyme or enzymes is or are introduced into the gel phase, in particular into the gel phase with gel former, not as a liquid enzyme preparation. It is particularly preferred that the liquid enzyme preparations are not introduced into the gel phases in which the gel former is selected from gelatin, xanthan gum and/or polyvinyl alcohol, in particular gelatin or polyvinyl alcohol, particularly preferably polyvinyl alcohol. The introduction of liquid enzyme preparations into gel phases still heated by the production process, in particular in the production of PVOH-containing gel phases in which high temperatures are used, leads to the stability of the enzymes in the liquid enzyme preparation being greatly reduced. A reduction in the stability means, in particular, that the activity of the enzymes is significantly reduced in comparison with the starting preparation. This reduction in stability can be measured, for example, by determining the residual enzyme activity or by comparing the cleaning performance of the corresponding enzymes, in particular according to IKW standards. The enzymes are particularly preferably incorporated into the gel phases as granules.

Surprisingly, PVOH has been demonstrated to be particularly suitable for preparing gel phases that meet the requirements set out above. At least one gel phase is therefore particularly preferred which, in addition to at least one enzyme, in particular at least one enzyme granulate, preferably selected from the group of proteases and/or amylases, has PVOH and at least one polyhydric alcohol. Particularly preferably, the at least one gel phase, at least one amylase granulate and/or at least one protease granulate, comprises PVOH and at least one polyhydric alcohol.

The at least one gel phase comprises at least one enzyme, preferably at least one enzyme granulate, preferably at least one amylase granulate and/or at least one protease granulate, in particular at least one protease granulate, PVOH and/or derivatives thereof in a proportion of from approximately 4 wt. % to 40 wt. %, in particular from 6 wt. % to 30 wt. %, preferably from 7 to 24 wt. %, particularly preferably between 8 wt. % to 22 wt. %. Significantly lower proportions of PVOH do not lead to the formation of a stable gel phase. The values are based in each case on the total weight of the gel phase.

According to a particularly preferred embodiment, the at least one gel phase comprises PVOH (polyvinyl alcohol). These gel phases prepared in this way are particularly high-melting, dimensionally stable (even at 40° C.) and do not change in shape during storage, or change only insignificantly. In particular, they are also less reactive with respect to a direct negative interaction with components of the granular mixture, in particular the powder phase. PVOH can in particular also produce low-water or water-free gel phases without difficulty. Using PVOH as the polymer for the at least one gel phase results in low-viscosity melts at 110-120° C. which can therefore be processed particularly easily, in particular the filling of the gel phase into the water-soluble wrapping can be carried out quickly and accurately without any adhesion or the quantity being inaccurately metered. Furthermore, said gel phases adhere particularly well to the water-soluble wrapping, in particular if it is also made of PVOH. This is also visually advantageous. Due to the rapid solidification of the at least one gel phase comprising PVOH, the further processing of the gel phases can take place particularly quickly. Furthermore, the good solubility of the gel phases produced is particularly favorable for the overall solubility of the cleaning agent. In addition, gel phases having such short solidification times are advantageous since the at least one solid phase, which is metered thereon and comprises granular mixtures, in particular powder, does not sink into the gel which is not yet completely set or is still too soft. This leads to cleaning agent portions that are less visually attractive.

In the multiphase single-use portions that comprise at least one solid phase, it is particularly important that the at least one second phase is dimensionally stable, so that as few interactions as possible can take place between the solid phase and the gel phase. If the at least one gel phase comprises gelatin in addition to PVOH, the tenacity of the gel phase during preparation is surprisingly increased.

A further preferred objective relates to cleaning agents, preferably dishwasher detergents, in particular automatic dishwasher detergents, which contain in the gel phase, in addition to the at least one enzyme, preferably the at least one enzyme granulate, at least one organic solvent, in particular selected from 1,2-propanediol, 1,3-propanediol, glycerol, 1,1,1-trimethylolpropane, triethylene glycol, dipropylene glycol, polyethylene glycols and/or mixtures thereof.

The at least one gel phase preferably comprises at least one polyhydric alcohol. In addition to the preparation of flowable gel phases, the at least one polyhydric alcohol enables the preparation of a dimensionally stable, non-flowable gel phase within a short setting time that is within 15 minutes or less, in particular 10 minutes or less. Polyhydric alcohols are hydrocarbons in which two, three or more hydrogen atoms are replaced by OH groups. The OH groups are each bonded to different carbon atoms. No carbon atom has two OH groups. This is in contrast with (simple) alcohols, in which only one hydrogen atom is replaced by an OH group in hydrocarbons. Polyhydric alcohols having two OH groups are referred to as alkanediols, and polyhydric alcohols having three OH groups are referred to as alkanetriols. A polyhydric alcohol thus corresponds to general formula [KW](OH)ₓ, where KW represents a hydrocarbon that is linear or branched, saturated or unsaturated, substituted or unsubstituted. A substitution can occur with —SH or —NH— groups, for example. Preferably, KW is a linear or branched, saturated or unsaturated, unsubstituted hydrocarbon. KW comprises at least two carbon atoms. The polyhydric alcohol comprises 2, 3 or more OH groups (x=2, 3, 4, . . . ), with only one OH group being bonded to each C atom of the KW. Particularly preferably, KW comprises 2 to 10, i.e., 2, 3, 4, 5, 6, 7, 8, 9 or 10, carbon atoms. Polyhydric alcohols in which x=2, 3, or 4 can be used in particular (for example, pentaerythritol where x=4). Preferably x=2 (alkanediol) and/or x=3 (alkanediol).

Particularly preferably, the at least one gel phase comprises at least one alkanetriol and/or at least one alkanediol, in particular at least one C₃ to C₁₀ alkanetriol and/or at least one C₃ to C₁₀ alkanediol, preferably at least one C₃ to C₈ alkanetriol and/or at least one C₃ to C₈ alkanediol, particularly at least one C₃ to C₆ alkanetriol and/or at least one C₃ to C₅ alkanediol, as a polyhydric alcohol. Preferably, it comprises one alkanetriol and one alkanediol as at least one polyhydric alcohol. In a preferred embodiment, the at least one gel phase thus comprises at least one polymer, in particular PVOH or PVOH with gelatin, as well as at least one alkanediol and at least one alkanetriol, in particular one alkanetriol and one alkanediol. A gel phase comprising at least one polymer, PVOH or PVOH with gelatin, and a C₃ to C₈ alkanediol and a C₃ to C₈ alkanetriol is equally preferred. A gel phase comprising at least one polymer, in particular PVOH or PVOH with gelatin, and a C₃ to C₅ alkanediol and a C₃ to C₆ alkanetriol is more preferred. The polyhydric alcohols do not comprise any derivatives thereof, such as ethers, esters, etc.

Surprisingly, it has been demonstrated that, when a corresponding triol (alkanetriol) is combined with a corresponding diol (alkanediol), particularly short setting times can be achieved. Furthermore, the gel phases that are obtained are transparent and have a shiny surface, which provides an attractive visual impression of the cleaning agent. The terms “diol” and “alkanediol” are used synonymously herein. The same applies to “triol” and “alkanetriol.”

According to a particularly preferred embodiment, the cleaning agents, preferably dishwasher detergents, in particular automatic dishwasher detergents, contain the at least one organic solvent in the gel phase in quantities of from 30 to 90 wt. %, in particular from 40 to 85 wt. %, more preferably from 50 to 80 wt. %, based on the total weight of the gel phase.

The quantity of polyhydric alcohol or polyhydric alcohols used in gel phases is preferably at least 45 wt. %, in particular 55 wt. % or more. Preferred quantity ranges are from 45 wt. % to 85 wt. %, in particular from 50 wt. % to 80 wt. %, based on the total weight of the gel phase.

Preferably, the C₃ to C₆ alkanetriol is glycerol and/or 2-ethyl-2-(hydroxymethyl)-1,3-propanediol (also called 1,1,1-trimethylolpropane) and/or 2-amino-2-(hydroxymethyl)-1,3-propanediol (TRIS, tris hydroxymethyl aminoethane) and/or 1,3,5-pentanetriol.

The C₃ to C₆ alkanetriol is particularly preferably glycerol and/or 2-ethyl-2-(hydroxymethyl)-1,3-propanediol (also called 1,1,1-trimethylolpropane). The C₃ to C₅ alkanediol is, for example, 1,5-pentanediol, 3-methyl-1,5-pentanediol, 1,4-butanediol, 1,3-propanediol and/or 1,2-propanediol, preferably 1,3-propanediol and/or 1,2-propanediol. Surprisingly, it has been found that the chain length of the diol as well as, in particular, the position of the OH groups has an influence on the transparency of the gel phase. The OH groups of the diol are therefore preferably not arranged on immediately adjacent C atoms. In particular, three or four carbon atoms, particularly preferably three carbon atoms, are located between the two OH groups of the diol. Particularly preferably, the diol is 1,3-propanediol. Surprisingly, it has been found that particularly good results are obtained with mixtures that comprise glycerol and 1,3-propanediol and/or 1,2-propanediol, in particular glycerol and 1,3-propanediol.

Polyethylene glycol(s) having an average molar mass of from 200 to 600 g/mol are preferably also used in the at least one gel phase or the gel phases. In combination with polyvinyl alcohol, polyethylene glycols with an average molar mass of between about 200 and about 600 g/mol, preferably between 300 and 500 g/mol, in particular between 350 and 450 g/mol, for example around 400 g/mol (INCI. PEG400), are used. Cleaning agent portions are therefore characterized in that they comprise polyethylene glycol(s) having an average molar mass of from 300 to 500 g/mol, in particular from 350 to 450 g/mol.

The at least one gel phase comprises at least one enzyme, preferably at least one enzyme granulate, preferably at least one amylase granulate and/or at least one protease granulate, in particular at least one protease granulate, PVOH and/or derivatives thereof, preferably in a proportion of from approximately 4 wt. % to 40 wt. %, in particular from 6 wt. % to 30 wt. %, preferably from 7 to 24 wt. %, particularly preferably between 8 wt. % to 22 wt. %, and at least one polyhydric alcohol, optionally additionally polyethylene glycols having an average molecular mass of from approximately 200 to 600 g/mol in amounts of from 5 to 30 wt. %, preferably from 8 to 26 wt. %, in particular from 10 to 22 wt. %, based on the total weight of the at least one gel phase.

Surprisingly, it has been found that the addition of polyethylene glycols, in particular those having average molar masses of from 200 to 600 g/mol, to the at least one gel phase, in particular in gel phases comprising polyvinyl alcohol, leads to an acceleration of the solidification time of the gel phases. Values of a few minutes and even less than a minute can be achieved. This is highly advantageous, in particular for the production sequences, since the further processing of the gel phases in the solidified state can take place much more quickly and therefore usually more cost-effectively. Surprisingly, it has been found that the presence of polyethylene glycol(s) having an average molar mass of from 200 to 600 g/mol in combination with polyvinyl alcohol and/or derivatives thereof contributes significantly to reducing the setting times. Without being bound to the theory, it is assumed that such polyethylene glycols, in particular those having a molar mass of from 350 to 450 g/mol, in particular approximately 400 g/mol, increase the sol-gel temperature.

In a particularly preferred embodiment, the quantity of polyethylene glycol(s) having an average molar mass of from 350 to 450 g/mol, for example approximately 400 g/mol, is 10 to 22 wt. % based on the total weight of the gel phase.

The at least one gel phase comprises at least one enzyme, preferably at least one enzyme granulate, preferably at least one amylase granulate and/or at least one protease granulate, in particular at least one protease granulate, PVOH and/or derivatives thereof in a proportion of from approximately 4 wt. % to 40 wt. %, in particular from 6 wt. % to 30 wt. %, preferably from 7 to 24 wt. %, particularly preferably between 8 wt. % to 22 wt. %, polyethylene glycol(s) having an average molar mass of from 200 to 600 g/mol and 1,3-propanediol and glycerol or 1,1,1-trimethylolpropane as polyhydric alcohols. A particularly preferred gel phase comprises gelatin or PVOH as a polymer and 1,3-propanediol and glycerin or 1,1,1-trimethylolpropane as polyhydric alcohols. If the gel phase, in each case based on the total weight of the gel phase, comprises an alkanetriol, in particular glycerin or 1,1,1-trimethylolpropane, then the proportion of alkanetriol, in particular glycerin or 1,1,1-trimethylolpropane, is between 3 and 75 wt. %, preferably 5 wt. % to 70 wt. %, in particular 10 wt. % to 65 wt. %, particularly 20 wt. % to 40 wt. %, based on the total weight of the gel phase.

If the gel phase optionally comprises a plurality of alkanetriol(s), then the total proportion of alkanetriol(s) is between 3 and 75 wt. %, preferably 5 wt. % to 70 wt. %, in particular 10 wt. % to 65 wt. %, particularly 20 wt. % to 40 wt. %, based on the total weight of the gel phase.

If glycerol is contained as an alkanetriol in the gel phase, then the proportion of glycerol based on the total weight of the gel phase is preferably 5 wt. % to 70 wt. %, particularly 10 wt. % to 65 wt. %, particularly 20 wt. % to 40 wt. %.

If 1,1,1-trimethylolpropane is contained in the gel phase, then the proportion of 1,1,1-trimethylolpropane based on the total weight of the gel phase is preferably 5 wt. % to 70 wt. %, in particular 10 wt. % to 65 wt. %, particularly preferably 18 to 45 wt. %, particularly preferably 20 wt. % to 40 wt. %.

If 2-amino-2-hydroxymethyl-1,3-propanediol is present in the gel phase, the proportion of 2-amino-2-hydroxymethyl-1,3-propanediol, based on the total weight of the gel phase, is preferably 5 wt. % to 70 wt. %, in particular 10 wt. % to 65 wt. %, in particular 20 wt. % to 40 wt. %.

If a plurality of alkanediols is optionally present in the gel phase, the proportion of alkanediols, based on the total weight of the gel phase, is preferably 5 wt. % to 70 wt. %, in particular 7 wt. % to 65 wt. %, particularly 10 wt. % to 40 wt. %.

If, in addition to at least one enzyme, preferably at least one enzyme granulate, preferably at least one amylase granulate and/or at least one protease granulate, in particular at least one subtilisin-containing protease granulate, the gel phase, in each case based on the total weight of the gel phase, comprises at least one alkanediol, in particular 1,3-propanediol or 1,2-propanediol, the proportion of alkanediol, in particular 1,3-propanediol or 1,2-propanediol, based on the total weight of the gel phase, is preferably 5 wt. % to 70 wt. %, in particular 10 wt. % to 65 wt. %, in particular 20 wt. % to 45 wt. %. If 1,3-propanediol is present in the gel phase, the proportion of 1,3-propanediol, based on the total weight of the gel phase, is in particular 10 wt. % to 65 wt. %, in particular 20 wt. % to 45 wt. %.

A gel phase is preferred which, based on the total weight of the gel phase, contains, in addition to at least one enzyme, preferably at least one enzyme granulate, preferably at least one amylase granulate and/or at least one protease granulate, in particular at least one subtilisin-containing protease granulate, 20 to 45 wt. % 1,3 propanediol and/or 1,2-propanediol and 10 wt. % to 65 wt. % 2-amino-2-hydroxymethyl-1,3-propanediol, in each case based on the total weight of the gel phase. Also preferred is a gel phase containing 20 to 45 wt. % of 1,3 propanediol and/or 1,2 propanediol and 10 to 65 wt. % of 1,1,1-trimethylolpropane, in each case based on the total weight of the gel phase. In particular, a gel phase containing 20 to 45 wt. % of 1,3 propanediol and/or 1,2 propanediol and 10 to 65 wt. % of glycerol, in each case based on the total weight of the gel phase, is preferred.

It was found that, in these ranges, quick setting of a gel phase is possible at 20° C. and yields phases that are stable in storage and transparent. In particular, the glycerol proportion has an impact on the curing time.

If the at least one gel phase, based in each case on the total weight of the gel phase, has, in addition to at least one enzyme, preferably at least one enzyme granulate, preferably at least one amylase granulate and/or at least one subtilisin-containing protease granulate, in particular at least one subtilisin-containing protease granulate, a C₃ to C₆ alkanetriol and a C₃ to C_{S} alkanediol, their weight ratio is preferably 3:1 to 1:2. In particular, the weight ratio is from 2:1 to 1:1.5, preferably from 1.5:1 to 1:1.2, preferably from 1.3 to 1:1, when glycerol and 1,3-propanediol are contained as polyhydric alcohols. Surprisingly, it has been found that, with these weight ratios, storage-stable, shiny, transparent gel phases can be obtained within short setting times of 10 minutes or less at 20° C. In combination with polyethylene glycols having an average molar mass of from 200 to 600 g/mol, the preferred above-mentioned weight ratios, in particular weight ratios (C₃ to C₆ alkanetriol:C₃ to C₅ alkanediol) of from 1.5:1 to 1:1.2, allow a reduction to setting times of 5 minutes or less.

According to a further preferred embodiment, in addition to the above-mentioned alkanols, triethylene glycol may be present in the at least one gel phase, in particular the gel phases described above as being preferred, especially if this phase contains PVOH and optionally polyethylene glycols having an average molar mass of from 200 to 600 g/mol. Triethylene glycol advantageously accelerates the solidification of the gel phase(s). It also causes the resulting gel phase to exchange little, if any, liquid with the environment, in a manner that is not perceptible. This improves in particular the visual impression of the resulting cleaning agent portions. It is particularly preferred if the at least one gel phase, in each case based on the total weight of the gel phase, contains, in addition to at least one enzyme, preferably at least one enzyme granulate, preferably at least one amylase granulate and/or at least one protease granulate, in particular at least one subtilisin-containing protease granulate, 1,3 and/or 1,2-propanediol, particularly preferably 1 to 3.5 wt. % 1,3-propanediol, and glycerol between 0.1 and 20 wt. %, preferably between 1 and 15 wt. %, in particular between 5 and 12 wt. %, for example 8 to 11 wt. % triethylene glycol.

Furthermore, the at least one gel phase preferably comprises another anionic polymer, in particular polycarboxylates. These can act either as builders and/or as thickening polymers. The at least one gel phase can further comprise anionic polymers or copolymers having builder properties. This is preferably a polycarboxylate. A copolymeric polyacrylate, preferably a sulfopolymer, preferably a copolymeric polysulfonate, preferably a hydrophobically modified copolymeric polysulfonate, is preferably used as the polycarboxylate. The copolymers can have two, three, four, or more different monomer units. Preferred copolymeric polysulfonates contain, besides sulfonic acid group-containing monomer(s), at least one monomer from the group of unsaturated carboxylic acids.

According to a particularly preferred embodiment, the low-water gel phase contains a polymer comprising at least one sulfonic acid group-containing monomer.

As unsaturated carboxylic acid(s), unsaturated carboxylic acids of formula R¹(R²)C═C(R³)COOH are particularly preferably used, in which R¹ to R³ represent, independently of one another, —H, —CH₃, a straight-chain or branched saturated alkyl functional group having 2 to 12 carbon atoms, a straight-chain or branched, mono- or polyunsaturated alkenyl functional group having 2 to 12 carbon atoms, —NH₂, —OH, or —COOH-substituted alkyl or alkenyl functional groups as defined above, or represent —COOH or —COOR⁴, where R⁴ is a saturated or unsaturated, straight-chain or branched hydrocarbon functional group having 1 to 12 carbon atoms.

Particularly preferred unsaturated carboxylic acids are acrylic acid, methacrylic acid, ethacrylic acid, α-chloroacrylic acid, α-cyanoacrylic acid, crotonic acid, α-phenylacrylic acid, maleic acid, maleic anhydride, fumaric acid, itaconic acid, citraconic acid, methylene malonic acid, sorbic acid, cinnamic acid, or mixtures thereof. Unsaturated dicarboxylic acids can of course also be used.

For sulfonic acid group-containing monomers, those of the formula R⁵(R⁶)C═C(R⁷)—X—SO₃H are preferred, in which R⁵ to R⁷, independently of one another, represent —H, —CH₃, a straight-chain or branched saturated alkyl functional group having 2 to 12 carbon atoms, a straight-chain or branched, mono- or polyunsaturated alkenyl functional group having 2 to 12 carbon atoms, —NH₂, —OH, or —COOH-substituted alkyl or alkenyl functional groups, or represent —COOH or —COOR⁴, where R⁴ is a saturated or unsaturated, straight-chain or branched hydrocarbon functional group having 1 to 12 carbon atoms, and X represents an optionally present spacer group that is selected from —(CH₂)ₙ—, where n=0 to 4, —COO—(CH₂)ₖ—, where k=1 to 6, —C(O)—NH—C(CH₃)₂—, —C(O)—NH—C(CH₃)₂—CH₂— and —C(O)—NH—CH(CH₂CH₃)—CH₂—.

Among these monomers, those of formulas H₂C═CH—X—SO₃H, H₂C═C(CH₃)—X—SO₃H or HO₃S—X—(R⁶)C═C(R⁷)—X—SO₃H are preferred, in which R⁶ and R⁷, independently of one another, are selected from —H, —CH₃, —CH₂CH₃, —CH₂CH₂CH₃ and —CH(CH₃)₂, and X represents an optionally present spacer group that is selected from —(CH₂)ₙ—, where n=0 to 4, —COO—(CH₂)ₖ—, where k=1 to 6, —C(O)—NH—C(CH₃)₂-, —C(O)—NH—C(CH₃)₂—CH₂— and —C(O)—NH—CH(CH₃)—CH₂—.

According to a particularly preferred embodiment, the gel phase comprises a polymer comprising, as a sulfonic acid group-containing monomer, acrylamidopropanesulfonic acids, methacrylamidomethylpropanesulfonic acids or acrylamidomethylpropanesulfonic acid.

Particularly preferred sulfonic acid group-containing monomers are 1-acrylamido-1-propanesulfonic acid, 2-acrylamido-2-propanesulfonic acid, 2-acrylamido-2-methyl-1-propanesulfonic acid, 2-methacrylamido-2-methyl-1-propanesulfonic acid, 3-methacrylamido-2-hydroxy-propanesulfonic acid, allyl sulfonic acid, methallyl sulfonic acid, allyloxybenzene sulfonic acid, methallyloxybenzene sulfonic acid, 2-hydroxy-3-(2-propenyloxy)propanesulfonic acid, 2-methyl-2-propene-1-sulfonic acid, styrene sulfonic acid, vinylsulfonic acid, 3-sulfopropylacrylate, 3-sulfopropylmethacrylate, sulfomethacrylamide, sulfomethylmethacrylamide, as well as mixtures of the above acids or water-soluble salts thereof. The sulfonic acid groups can be present in the polymers in a fully or partially neutralized form, i.e., the acidic hydrogen atom of the sulfonic acid group can be replaced in some or all of the sulfonic acid groups with metal ions, preferably alkali metal ions, and in particular with sodium ions. The use of partially or fully neutralized sulfonic acid group-containing copolymers is preferred.

In copolymers that contain only carboxylic acid group-containing monomers and sulfonic acid group-containing monomers, the monomer distribution of the copolymers that are preferably used is preferably 5 to 95 wt. % in each case; particularly preferably, the proportion of the sulfonic acid group-containing monomer is 50 to 90 wt. %, and the proportion of the carboxylic acid group-containing monomer is 10 to 50 wt. %, with the monomers preferably being selected from those mentioned above. The molar mass of the sulfo-copolymers that are preferably used can be varied in order to adapt the properties of the polymers to the desired intended use. Preferred cleaning agents are characterized in that the copolymers have molar masses of from 2,000 to 200,000 gmol⁻¹, preferably 4,000 to 25,000 gmol⁻¹, and in particular 5,000 to 15,000 gmol⁻¹.

In another preferred embodiment, the copolymers comprise not only a carboxyl group-containing monomer and sulfonic acid group-containing monomer but also at least one non-ionic, preferably hydrophobic monomer. In particular, the rinsing performance of dishwasher detergents was able to be improved through the use of these hydrophobically modified polymers.

Particularly preferably, the at least one gel phase further comprises an anionic copolymer, a copolymer comprising
i) carboxylic acid group-containing monomers
ii) sulfonic acid group-containing monomers
iii) non-ionic monomers, in particular hydrophobic monomers, being used as the anionic copolymer.

As non-ionic monomers, monomers of general formula R¹(R²)C═C(R³)—X—R⁴ are preferably used, in which R¹ to R³ represent, independently of one another, —H, —CH₃ or —C₂Hs, X represents an optionally present spacer group selected from —CH₂—, —C(O)O— and —C(O)—NH—, and R⁴ represents a straight-chain or branched saturated alkyl functional group having 2 to 22 carbon atoms or an unsaturated, preferably aromatic functional group having 6 to 22 carbon atoms.

Particularly preferred non-ionic monomers are butene, isobutene, pentene, 3-methylbutene, 2-methylbutene, cyclopentene, hexene, hexene-1, 2-methlypentene-1, 3-methlypentene-1, cyclohexene, methylcyclopentene, cycloheptene, methylcyclohexene, 2,4,4-trimethylpentene-1,2,4,4-trimethylpentene-2,2,3-dimethylhexene-1,2,4-dimethylhexene-1,2,5-dimethylhexene-1,3,5-dimethylhexene-1,4,4-dimethylhexane-1, ethylcyclohexene, 1-octene, α-olefins having 10 or more carbon atoms such as 1-decene, 1-dodecene, 1-hexadecene, 1-octadecene and C₂₂ α-olefin, 2-styrene, α-methylstyrene, 3-methylstyrene, 4-propylstyrene, 4-cyclohexylstyrene, 4-dodecylstyrene, 2-ethyl-4-benzylstyrene, 1-vinyl naphthalene, 2-vinyl naphthalene, acrylic acid methyl ester, acrylic acid ethyl ester, acrylic acid propyl ester, acrylic acid butyl ester, acrylic acid pentyl ester, acrylic acid hexyl ester, methacrylic acid methyl ester, N-(methyl)acrylamide, acrylic acid-2-ethylhexyl ester, methacrylic acid-2-ethylhexyl ester, N-(2-ethylhexyl)acrylamide, acrylic acid octyl ester, methacrylic acid octyl ester, N-(octyl)acrylamide, acrylic acid lauryl ester, methacrylic acid lauryl ester, N-(lauryl)acrylamide, acrylic acid stearyl ester, methacrylic acid stearyl ester, N-(stearyl)acrylamide, acrylic acid behenyl ester, methacrylic acid behenyl ester, and N-(behenyl)acrylamide or mixtures thereof, in particular acrylic acid, ethyl acrylate, 2-acrylamido-2-methylpropanesulfonic acid (AMPS) as well as mixtures thereof.

Surprisingly, PVOH and/or derivatives thereof together with anionic polymers or copolymers, in particular with sulfonic acid group-containing copolymers, have also been found to lead to the formation of gel phases with insensitive surfaces. Such surfaces can be touched by an end consumer without having material adhere to their hands. No erosion of material occurs in packaging, either. The gel phase therefore preferably comprises, in addition to at least one enzyme, preferably at least one enzyme granulate, PVOH, polyethylene glycol(s) having an average molar mass of 200 to 600 g/mol, at least one polyhydric alcohol, and an anionic copolymer/polymer. The proportion of the anionic polymer is preferably 1 wt. % to 35 wt. %, in particular 3 wt. % to 30 wt. %, in particular 4 wt. % to 25 wt. %, preferably 5 wt. % to 20 wt. %, for example 10 wt. %, based on the total weight of the gel phase. Sulfopolymers, in particular the preferred copolymeric polysulfonates, which, in addition to sulfonic acid group-containing monomer(s), also contain at least one monomer from the group of unsaturated carboxylic acids, in particular acrylic acid, also provide an excellent shine on the surface. What is more, fingerprints are not left behind. Therefore, the proportion of sulfopolymers, in particular the preferred copolymeric polysulfonates which contain not only sulfonic acid group-containing monomer(s) but also at least one monomer from the group of unsaturated carboxylic acids, in particular acrylic acid, in particular the proportion of said sulfopolymers having AMPS as a sulfonic acid group-containing monomer, for example Acusol 590, Acusol 588 or Sokalan CP50, is preferably 1 wt. % to 25 wt. %, in particular 3 wt. % to 18 wt. %, particularly 4 wt. % to 15 wt. %, preferably 5 wt. % to 12 wt. %, based on the weight of the gel phase. In a particularly preferred embodiment, the at least one gel phase, in addition to at least one enzyme, preferably at least one enzyme granulate, preferably at least one amylase granulate and/or at least one protease granulate, in particular at least one subtilisin-containing protease granulate, therefore comprises PVOH and a sulfopolymer, in particular the preferred copolymeric polysulfonates which contain not only sulfonic acid group-containing monomer(s) but also at least one monomer from the group of unsaturated carboxylic acids, in particular acrylic acid, and at least one polyhydric alcohol.

According to another embodiment, in addition to said polyethylene glycols having an average molar mass of from 200 to 600 g/mol, further polyalkylene glycols, in particular further polyethylene glycols, having an average molar mass of between approximately 800 and 8,000, may be contained in the at least one gel phase. The above-mentioned polyethylene glycols are particularly preferably used in quantities of from 1 to 40 wt. %, preferably from 5 to 35 wt. %, in particular from 10 to 30 wt. %, for example from 15 to 25 wt. %, preferably in each case based on the total weight of the gel phase.

Particularly preferred embodiments comprise, as at least one gel phase, in each case based on the total weight of the gel phase, in addition to at least one enzyme, preferably at least one enzyme granulate, preferably at least one amylase granulate and/or at least one protease granulate, in particular at least one subtilisin-containing protease granulate, 8 to 22 wt. % PVOH, 15 to 40 wt. % 1,3-propanediol, 20 to 40 wt. % glycerol, 5 to 15 wt. % sulfonic acid group-containing polyacrylate copolymer, and 8 to 22 wt. %, in particular 10 to 20 wt. %, polyethylene glycol having an average molar mass of 200-600 g/mol, optionally 2 to 10 wt. % 1,2-propanediol, and optionally also 2-15 wt. % triethylene glycol, in each case based on the total weight of the gel phase.

According to another particularly preferred embodiment, the cleaning agent, preferably dishwasher detergent, in particular automatic dishwasher detergent, is a cleaning agent portion in a water-soluble wrapping having one or more chambers/compartments. The cleaning agent is preferably packaged as an individual cleaning agent portion, such that it is used to carry out a dishwasher cycle and is (as far as possible) substantially consumed in the process.

The water-soluble wrapping is preferably made from a water-soluble film material, which is selected from the group consisting of polymers or polymer mixtures. The wrapping may be made up of one or of two or more layers of the water-soluble film material. The water-soluble film material of the first layer and of the additional layers, if present, may be the same or different.

It is preferable for the water-soluble wrapping to contain polyvinyl alcohol or a polyvinyl alcohol copolymer. Water-soluble wrappings containing polyvinyl alcohol or a polyvinyl alcohol copolymer exhibit good stability with a sufficiently high level of water solubility, in particular cold-water solubility.

Suitable water-soluble films for producing the water-soluble wrapping are preferably based on a polyvinyl alcohol or a polyvinyl alcohol copolymer of which the molecular weight is in the range of from 10,000 to 1,000,000 gmol⁻¹, preferably from 20,000 to 500,000 gmol⁻¹, particularly preferably from 30,000 to 100,000 gmol⁻¹ and in particular from 40,000 to 80,000 gmol⁻¹.

Polyvinyl alcohol is usually prepared by hydrolysis of polyvinyl acetate, since the direct synthesis route is not possible. The same applies to polyvinyl alcohol copolymers, which are prepared accordingly from polyvinyl acetate copolymers. It is preferable for at least one layer of the water-soluble wrapping to include a polyvinyl alcohol of which the degree of hydrolysis is 70 to 100 mol. %, preferably 80 to 90 mol. %, particularly preferably 81 to 89 mol. %, and in particular 82 to 88 mol. %.

In a preferred embodiment, the water-soluble packaging consists of at least 20 wt. %, particularly preferably at least 40 wt. %, very particularly preferably at least 60 wt. %, and in particular at least 80 wt. % of a polyvinyl alcohol of which the degree of hydrolysis is 70 to 100 mol. %, preferably 80 to 90 mol. %, particularly preferably 81 to 89 mol. %, and in particular 82 to 88 mol. %.

In addition, a polymer selected from the group comprising (meth)acrylic acid-containing (co)polymers, polyacrylamides, oxazoline polymers, polystyrene sulfonates, polyurethanes, polyesters, polyethers, polylactic acid or mixtures of said polymers may be added to a polyvinyl alcohol-containing film material that is suitable for producing the water-soluble wrapping. Polylactic acids are a preferred additional polymer.

Preferred polyvinyl alcohol copolymers include, in addition to vinyl alcohol, dicarboxylic acids as further monomers. Suitable dicarboxylic acids are itaconic acid, malonic acid, succinic acid and mixtures thereof, with itaconic acid being preferred. Polyvinyl alcohol copolymers which include, in addition to vinyl alcohol, an ethylenically unsaturated carboxylic acid, or the salt or ester thereof, are also preferred. Polyvinyl alcohol copolymers of this kind particularly preferably contain, in addition to vinyl alcohol, acrylic acid, methacrylic acid, acrylic acid ester, methacrylic acid ester or mixtures thereof.

It may be preferable for the film material to contain further additives. The film material may contain plasticizers such as dipropylene glycol, ethylene glycol, diethylene glycol, propylene glycol, glycerol, sorbitol, mannitol or mixtures thereof, for example. Further additives include for example release aids, fillers, cross-linking agents, surfactants, antioxidants, UV absorbers, anti-blocking agents, anti-adhesive agents or mixtures thereof.

Suitable water-soluble films for use in the water-soluble wrappings of the water-soluble packaging are films which are sold by MonoSol LLC, for example under the names M8720, M8630, M8312, M8440, M7062, C8400 or M8900. Films which are sold under the names SH2601, SH2504, SH2707 or SH2701 by Nippon Gohsei are also suitable. Other suitable films include films having the names Solublon® PT, Solublon® GA, Solublon® KC or Solublon® KL from Aicello Chemical Europe GmbH, or the VF-HP films from Kuraray.

The water-soluble wrapping preferably has, at least in part, a bittern having a bitter value of between 1,000 and 200,000, in particular those selected from quinine sulfate (bitter value=10,000), naringin (bitter value=10,000), sucrose octaacetate (bitter value=100,000), quinine hydrochloride and mixtures thereof. In particular, the outer surface of the water-soluble wrapping is coated at least in part with a bittern having a bitter value of between 1,000 and 200,000. In this connection, it is particularly preferable for the water-soluble wrapping to be coated by at least 50%, preferably at least 75%, and very particularly preferably at least 90%, with the bittern having a bitter value of between 1,000 and 200,000. The application of the bittern having a bitter value of between 1,000 and 200,000 can take place for example by means of printing, spraying or spreading.

The water-soluble wrapping has at least one continuous peripheral sealing seam that lies substantially in one plane. This is favorable from a process point of view since only a single sealing step, possibly using only a single sealing tool, is required for a peripheral sealing seam that lies substantially in one plane. The continuous circumferential sealing seam leads to better closure compared with those wrappings having a plurality of sealing seams and to excellent sealing of the sealed seam and thus of the wrapping itself. Product leaking out of the wrapping, e.g., onto the surface of the portion, would be detrimental as the consumer would then come into contact with the product. Precisely this situation should be avoided as far as possible with a cleaning agent portion comprising a water-soluble wrapping.

The water-soluble wrapping may preferably be produced from at least two packaging parts. Preferably, the at least two packaging parts are water-soluble, so that no packaging parts remain in the dishwasher, which can then lead to problems in the dishwasher. The at least two packaging parts do not have to be different. They may preferably be produced from the same material and in the same way. In a preferred embodiment, these are two parts of a water-soluble film, in particular two parts of a water-soluble film of the same composition.

In another embodiment, the at least two packaging parts can be made of different materials, e.g. of different films or of material having two different properties (e.g. film that is soluble in hot and cold water). In this embodiment, it is preferable for a water-soluble film and another packaging part made by injection molding to be combined.

According to a particularly preferred embodiment, the water-soluble wrapping comprises at least one at least partly plastically deformed film. In particular, this plastic deformation of the film can be produced by methods known to a person skilled in the art, such as thermoforming (with and without application of a vacuum), blowing or stamping. In particular, the water-soluble wrapping comprises at least one at least partly plastically deformed film which has been produced by thermoforming.

The at least one solid phase and the at least one gel phase can be arranged within the water-soluble wrapping in any combination with one another. A solid phase can thus be arranged on or beside a gel phase. In this embodiment, the cleaning agent comprises a solid phase and a gel phase. It is also conceivable for a solid phase to be surrounded by gel phases. The embedding of one phase in another is also covered. In a further, particularly preferred arrangement, the gel phase is in cast form, for example in the form of a gel core, and is surrounded by the solid phase. Two or more cavities that are separated from one another can also be present which are filled with the at least one gel phase. In this embodiment, the cleaning agent comprises in particular two gel phases, it being possible for the two gel phases to have different compositions.

According to a preferred embodiment, three, four, five or six or more cavities that are separated from one another are present which are filled with one or more of the gel phases. Preferably, such cleaning agents comprise three, four, five or six or more gel phases, it being possible for said gel phases to have the same or also different compositions.

A preferred object relates to a cleaning agent, preferably a dishwasher detergent, in particular an automatic dishwasher detergent, which additionally comprises at least one solid, in particular particulate phase and optionally at least one other liquid/gel or solid phase.

“Solid” in this context means that the composition is in solid form under standard conditions (temperature 25° C., pressure 1013 mbar). Suitable solid phases are, in particular, granular mixtures of a solid composition, such as powders and/or granulate, in particular powder phases. Solid compositions/phases which have an increased dimensional stability in comparison with the loose granular mixture, in particular powder, for example powder or granular preparations which have been compacted by compression before or after introduction into the film, e.g., by restoring forces of the film after thermoforming, or also directly compressed compositions, such as compressed products or tablets, are also suitable. This at least one solid phase can be in direct contact with the gel phase. Cleaning agent portions, in particular multi-chamber bags, in which the solid and gel phases are spatially close to one another but separate from one another, are also used. The two chambers can for example be separated by a film, in particular a water-soluble film, or by a sealed seam (preferably a sealed seam of 3 mm or less). The chambers of a multi-chamber pouch are therefore on top of one another as well as beside one another. Furthermore, mixtures of single-chamber or multi-chamber bags may include a gel phase and, separated therefrom, at least one solid phase, which come into contact by arrangement, e.g., by folding and fixing a pouch, or by storage at a distance of less than 3 mm, for example in a packaging bag or a device for portioned metering.

The powder phase is understood to be a granular mixture. The term powder phase covers powders and/or granulate according to this definition.

A granular mixture is formed from a large number of loose, solid particles, which in turn comprise what are known as grains.

A grain is a name for the particulate constituents of powders (grains are the loose, solid particles), dusts (grains are the loose, solid particles), granulates (loose, solid particles are agglomerates of several grains), and other granular mixtures.

A preferred embodiment of the granular mixture of the composition of the solid phase is the powder and/or the granulate; when reference is made to “powder” or “granulate,” this also includes these being mixtures of different powders or different granulates. Accordingly, powder and granulate are also intended to mean mixtures of different powders with different granulates. Said solid particles of the granular mixture in turn preferably have a particle diameter X_{50.3} (volume average) of from 10 to 1,500 μm, more preferably from 200 μm to 1,200 μm, particularly preferably from 600 μm to 1,100 μm. Said particle sizes can be determined by sieving or by means of a Camsizer particle size analyzer from the company Retsch. The granular mixture of the solid composition serving as a solid phase is preferably present in free-flowing form (especially as free-flowing powder and/or free-flowing granulate). The average of the portion therefore comprises at least one solid phase of a free-flowing granular mixture of a solid composition, in particular a powder, and at least one gel phase as previously defined.

A particularly preferred objective relates to cleaning agents, in particular cleaning agent portions, in which the gel phase is in direct contact, for example in a chamber containing at least one solid phase.

Moreover, it is preferable for the at least one solid phase and the at least one gel phase to be in direct contact with one another. In this case, there should be substantially no interaction, in particular no negative interaction, between the solid phase and the gel phase. What substantially no negative interaction means in particular is that substantially no ingredients or solvents (less than 20 wt. %, preferably less than 10 wt. %, in particular less than 5 wt. %) go from one phase into the other or that the stability, in particular storage stability, preferably the stability at 4 weeks' storage and at a storage temperature of 30° C., in particular the cleaning performance after this storage, is reduced after this storage by less than 30%, preferably less than 20%, in particular less than 10%, and/or the aesthetics of the product are not impaired in an obvious way, for example through a change in color, the formation of moist-looking edges, a blurred boundary between the two phases, or the like. What no negative interaction means here, for example, is that no ingredients or solvents go from one phase into the other or that the stability, in particular storage stability, preferably the stability at 4 weeks' storage and at a storage temperature of 30° C., in particular with regard to the cleaning performance, and/or the aesthetics of the product are not impaired in any way, for example through a change in color, the formation of moist-looking edges, a blurred boundary between the two phases, or the like.

Surprisingly, it has been shown that by formulating a gel phase, preferably a dimensionally stable gel phase, comprising at least one enzyme, preferably at least one enzyme granulate, preferably at least one amylase granulate and/or at least one protease granulate, in particular at least one subtilisin-containing protease granulate, which is combined with a granular mixture of a solid composition, in particular a powder phase, this objective can be achieved. It is particularly suitable for the granular mixture of a solid composition, in particular the powder phase, to be free-flowing, because, owing to the process, it is possible to fill the water-soluble wrapping in a more targeted manner, in particular when filling a cavity produced by thermoforming. In addition, the visual appearance of the granular mixture of a solid composition, in particular the powder, can be better changed compared with a compressed tablet; in particular texture differences, such as coarse and fine particles and particles or regions having different colors—in full or as colored flecks—can be used to improve a visually pleasing appearance. In addition, the granular mixture of the solid composition, in particular the powder, offers improved solubility in comparison with compressed tablets, even without the addition of disintegrants.

A phase is a spatial region in which physical parameters and the chemical composition are homogeneous. One phase differs from another phase through its different features, such as ingredients, physical properties, external appearance, etc. Preferably, different phases can be differentiated visually from one another. The at least one solid phase can thus be clearly distinguished by a consumer from the at least one gel phase. If the cleaning agent has more than one solid phase, then said phases can also each be distinguished from one another with the naked eye because of their different coloration, for example. The same applies if two or more gel phases are present. In this case as well, a visual differentiation of the phases, for example on the basis of a difference in coloration or transparency, is possible. Phases are thus self-contained regions that can be differentiated visually from one another by a consumer with the naked eye. The individual phases can have different properties when used, such as the speed at which the phase dissolves in water and hence the speed and the sequence of the release of the ingredients contained in the particular phase.

The at least one solid phase comprises a granular mixture of a solid composition; in particular, it is powdery and free-flowing. The cleaning agent thus comprises at least one solid powder and free-flowing phase, as well as at least one gel phase, comprising at least one enzyme, preferably at least one enzyme granulate, preferably at least one amylase granulate and/or at least one protease granulate, in particular at least one subtilisin-containing protease granulate, and at least one polyvinyl alcohol, as gelling agent, comprises at least PVOH and/or derivatives thereof, particularly preferably at least PVOH, and at least one polyhydric alcohol.

The free-flowing ability of a granular mixture, in particular a powder solid, the powder phase, preferably the powder and/or granulate, relates to its ability to flow freely under its own weight. The free-flowing ability is determined by the outflow time of 1,000 ml of cleaning agent powder out of a standardized flow-test funnel, which is initially closed in its discharge direction and has an outlet of 16.5 mm in diameter, being measured by measuring the time for the complete outflow of the granular mixture, in particular the powder phase, preferably the powder and/or granulate, e.g. the powder, after opening the outlet, and being compared with the outflow speed (in seconds) of a standard test sand of which the outflow speed is defined as 100%. The defined sand mixture for calibrating the flow apparatus is dry sea sand. Sea sand having a particle diameter of 0.4 to 0.8 mm is used, available for example from Carl Roth, Germany CAS No. [14808-60-7]. For drying, the sea sand is dried before the measurement for 24 hours at 60° C. in a drying cabinet on a plate at a maximum layer height of 2 cm.

Preferred embodiments of the solid phases have an angle of repose/angle of slope of from 26 to 35, from 27 to 34, from 28 to 33, the angle of repose being determined according to the method mentioned below after 24 h following the preparation of the granular mixture of the solid composition, in particular the powder solid phase, preferably the powder and/or granulate, and storage at 20° C. Such angles of repose have the advantage that the cavities can be filled with the at least one solid phase comparatively quickly and precisely.

To determine the angle of repose (also referred to as the angle of slope) of the at least one solid phase, a powder funnel having a capacity of 400 ml and an outlet having a diameter of 25 mm is simply suspended in a tripod. The funnel is moved upwards by means of a manually operated knurling wheel at a speed of 80 mm/min such that the granular mixture, in particular the powder phase, preferably the powder and/or granulate, e.g., the powder, flows out. As a result, what is known as a conical heap is formed. The conical heap height and the conical heap diameter are determined for the individual solid phases. The angle of slope is calculated from the quotient of the conical heap height and the conical heap diameter*100.

Granular mixtures of a solid composition, in particular powder phases, preferably the powders and/or granulate, e.g., the powders, having a free-flowing ability in %, compared to the above-mentioned standard test substance, of greater than 40%, preferably greater than 50, in particular greater than 55%, more preferably greater than 60%, particularly preferably between 63% and 80%, for example between 65% and 75%, are particularly suitable. Granular mixtures of a solid composition, in particular powders and/or granulates having a free-flowing ability in %, compared to the above-mentioned standard test substance, of greater than 40%, preferably greater than 45%, in particular greater than 50%, more preferably greater than 55%, particularly preferably greater than 60%, are particularly suitable, the free-flowing ability being measured 24 hours following the preparation of the powder and storage at 20° C.

Lower values for the free-flowing ability are rather unsuitable, since from a process point of view, precise metering of the granular mixture, in particular the particulate phase, preferably the powder and/or granulate, e.g., the powder, is necessary. In particular, the values greater than 50%, in particular greater than 55%, preferably greater than 60% (where the measurement of the free-flowing ability is carried out 24 hours after the preparation of the powder and storage at 20° C.) have proved to be advantageous, since the good metering ability of the granular mixture, in particular the powder phases, preferably the powders and/or granulates, e.g. powder, leads to only minor fluctuations in the metered quantity or the composition. The more accurate metering leads to consistent product performance, and economic losses due to over-metering are thus avoided. It is further advantageous for the granular mixture, in particular the powder phase, preferably the powder and/or granulate, e.g., the powder, to be well metered so that a faster sequence of the metering process can be achieved. In addition, such a good free-flowing ability makes it easier to avoid the situation whereby the granular mixture, in particular the powder phase, preferably the powder and/or granulate, e.g., the powder, reaches the part of the water-soluble wrapping which is provided for producing the sealing seam and therefore ought to remain as free as possible of grains, in particular powder-free.

The granular mixture of the solid composition serving as a solid phase is preferably present in free-flowing form (especially as free-flowing powder and/or free-flowing granulate). The agent in the portion therefore comprises at least one solid phase of a free-flowing granular mixture of a solid composition, in particular a powder, and at least one gel phase as previously defined. The cleaning agent preferably comprises at least one surfactant. This surfactant is selected from the group of anionic, non-ionic, and cationic surfactants. The cleaning agent can also contain mixtures of several surfactants that are selected from the same group.

The at least one solid phase and/or the at least one gel phase comprise at least one surfactant. It is possible for only the at least one solid phase or only the at least one gel phase to comprise at least one surfactant. If both phases comprise a surfactant, then they are preferably surfactants that are different from one another. However, it is also possible that solid and gel phases have the same surfactant or surfactants. At least one solid and/or gel phase preferably contains at least one non-ionic surfactant. All non-ionic surfactants that are known to a person skilled in the art can be used as non-ionic surfactants. Low foaming non-ionic surfactants are preferably used, in particular alkoxylated, especially ethoxylated, low-foaming non-ionic surfactants such as alkyl glycosides, alkoxylated, preferably ethoxylated or ethoxylated and propoxylated fatty acid alkyl esters, polyhydroxy fatty acid amides, or amine oxides. Particularly preferred non-ionic surfactants are specified in greater detail below.

Preferred alcohol ethoxylates have a narrowed homolog distribution (narrow range ethoxylates, NRE). In addition to these non-ionic surfactants, fatty alcohols having more than 12 EO can also be used. Examples of these are tallow fatty alcohols having 14 EO, 25 EO, 30 EO, or 40 EO.

Ethoxylated non-ionic surfactants are particularly preferably used which were obtained from C₆₋₂₀ monohydroxy alkanols or C₆₋₂₀ alkyl phenols or C₁₆₋₂₀ fatty alcohols and more than 12 mol, preferably more than 15 mol, and in particular more than 20 mol, ethylene oxide per mol of alcohol. A particularly preferred non-ionic surfactant is obtained from a straight-chain fatty alcohol having 16 to 20 carbon atoms (C₁₆₋₂₀ alcohol), preferably from a C₁₈ alcohol and at least 12 mol, preferably at least 15 mol and in particular at least 20 mol of ethylene oxide. Of these, what are referred to as “narrow range ethoxylates” are particularly preferred.

Surfactants that are preferably used come from the group of the alkoxylated non-ionic surfactants, in particular the ethoxylated primary alcohols and mixtures of these surfactants with structurally complex surfactants such as polyoxypropylene/polyoxyethylene/polyoxypropylene ((PO/EO/PO) surfactants). Such (PO/EO/PO) non-ionic surfactants are also characterized by good foam control.

Low-foaming non-ionic surfactants which have alternating ethylene oxide and alkylene oxide units have proven to be particularly preferred.

Among these, in turn, surfactants having EO-AO-EO-AO blocks are preferred, with one to ten EO groups or AO groups being bonded to one another before a block of the other group follows. Here, non-ionic surfactants of the general formula

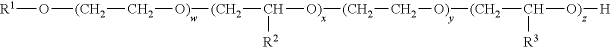

are preferred, in which R¹ represents a straight-chain or branched, saturated or mono- or polyunsaturated C₆₋₂₄-alkyl or alkenyl functional group; each R₂ and R₃ group is selected, independently of one another, from —CH₃, —CH₂CH₃, —CH₂CH₂—CH₃, —CH(CH₃)₂; and the indices w, x, y and z represent, independently of one another, integers from 1 to 6.

Preferred non-ionic surfactants of the above formula can be prepared using known methods, from the corresponding alcohols R¹—OH and ethylene or alkylene oxide. The R¹ functional group in the above formula can vary depending on the origin of the alcohol. If native sources are used, the R¹ functional group has an even number of carbon atoms and is generally unbranched, with the linear functional groups of alcohols of native origin having 12 to 18 C atoms, such as coconut, palm, tallow fatty or oleyl alcohol, for example, being preferred. Some examples of alcohols that are available from synthetic sources are the Guerbet alcohols or functional groups that are methyl-branched or linear and methyl-branched in the 2 position in admixture, such as those usually present in oxo alcohol functional groups. Irrespective of the type of alcohol used to prepare the non-ionic surfactants contained in the agents, non-ionic surfactants are preferred in which R¹ represents an alkyl functional group having 6 to 24, preferably 8 to 20, particularly preferably 9 to 15, and in particular 9 to 11, carbon atoms in the above formula.

Besides propylene oxide, butylene oxide in particular is worthy of consideration as an alkylene oxide unit that is contained alternately with the ethylene oxide unit in the preferred non-ionic surfactants. However, other alkylene oxides in which R² and R³ are selected, independently of one another, from —CH₂CH₂—CH₃ and —CH(CH₃)₂ are also suitable. Preferably, non-ionic surfactants of the above formula are used in which R² and R³ represent a —CH₃ functional group; w and x represent, independently of one another, values of 3 or 4; and y and z represent, independently of one another, values of 1 or 2.

Further preferably used non-ionic surfactants of the solid phase are non-ionic surfactants of general formula R¹O(AlkO)ₓM(OAlk)_{y}OR², in which R¹ and R² represent, independently of one another, a branched or unbranched, saturated or unsaturated, optionally hydroxylated alkyl functional group having 4 to 22 carbon atoms; Alk represents a branched or unbranched alkyl functional group having 2 to 4 carbon atoms; x and y represent, independently of one another, values of between 1 and 70; and M represents an alkyl functional group from the group CH₂, CHR³, CR³R⁴, CH₂CHR³ and CHR³CHR⁴, R³ and R⁴ representing, independently of one another, a branched or unbranched, saturated or unsaturated alkyl functional group having 1 to 18 carbon atoms.

Preferred in this case are non-ionic surfactants of general formula R¹—CH(OH)CH₂—O(CH₂CH₂O)ₓCH₂CHR(OCH₂CH₂)_{y}—CH₂CH(OH)—R², in which R, R¹ and R² represent, independently of one another, an alkyl functional group or alkenyl functional group having 6 to 22 carbon atoms; x and y represent, independently of one another, values between 1 and 40.

Preferred in this case are, in particular, compounds of general formula R¹—CH(OH)CH₂—O(CH₂CH₂O)ₓCH₂CHR(OCH₂CH₂)_{y}O—CH₂CH(OH)—R², in which R represents a linear, saturated alkyl functional group having 8 to 16 carbon atoms, preferably 10 to 14 carbon atoms, and n and m represent, independently of one another, values of from 20 to 30. Such compounds can be obtained, for example, by reacting alkyl diols HO—CHR—CH₂—OH with ethylene oxide, a reaction with an alkyl epoxide being performed subsequently in order to close the free OH functions during formation of a dihydroxy ether.

In this case, preferred non-ionic surfactants are those of general formula R¹—CH(OH)CH₂O-(AO)_{w}-(AO)ₓ-(A″O)_{y}-(A′″O)_{z}—R², in which
R¹ represents a straight-chain or branched, saturated or mono- or polyunsaturated C₆₋₂₄ alkyl or alkenyl functional group;
R² represents hydrogen or a linear or branched hydrocarbon functional group having 2 to 26 carbon atoms;
A, A′, A″ and A′″ represent, independently of one another, a functional group from the group —CH₂CH₂, —CH₂CH₂—CH₂, —CH₂—CH(CH₃), —CH₂—CH₂—CH₂—CH₂, —CH₂—CH(CH₃)—CH₂—, —CH₂—CH(CH₂—CH₃);
w, x, y and z represent values of between 0.5 and 120, where x, y and/or z can also be 0.

By adding the above-mentioned non-ionic surfactants of general formula R¹—CH(OH)CH₂O-(AO)_{w}-(A′O)ₓ-(A″O)_{y}-(A′″O)_{z}—R², subsequently also referred to as “hydroxy mixed ethers,” surprisingly, the cleaning performance of preparations can be significantly improved, both in comparison with surfactant-free systems and in comparison with systems containing alternative non-ionic surfactants, for example from the group of polyalkoxylated fatty alcohols.

By using these non-ionic surfactants having one or more free hydroxyl groups on one or both terminal alkyl functional groups, the stability of the enzymes contained in the cleaning agent preparations can be improved substantially.

In particular, those end-capped poly(oxyalkylated) non-ionic surfactants are preferred which, according to the following formula,

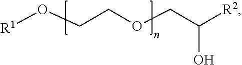

besides a functional group R¹, which represents linear or branched, saturated or unsaturated, aliphatic or aromatic hydrocarbon functional groups having 2 to 30 carbon atoms, preferably having 4 to 22 carbon atoms, also have a linear or branched, saturated or unsaturated, aliphatic or aromatic hydrocarbon functional group R² having 1 to 30 carbon atoms, where n represents values of between 1 and 90, preferably values of between 10 and 80, and in particular values of between 20 and 60. Surfactants of the above formula are in particular preferred in which R¹ represents C₇ to C₁₃, n represents a whole natural number from 16 to 28 and R² represents C₈ to C₁₂.

Surfactants of formula R¹O[CH₂CH(CH₃)O]ₓ[CH₂CH₂O]_{y}CH₂CH(OH)R² are particularly preferred, in which R¹ represents a linear or branched aliphatic hydrocarbon functional group having 4 to 18 carbon atoms or mixtures thereof, R² denotes a linear or branched hydrocarbon functional group having 2 to 26 carbon atoms or mixtures thereof, x represents values between 0.5 and 1.5, and y represents a value of at least 15. The group of these non-ionic surfactants includes for example the C₂₋₂₆ fatty alcohol (PO)₁-(EO)₁₅₋₄₀-2-hydroxyalkyl ethers, in particular including the C₈₋₁₀ fatty alcohol (PO)₁-(EO)₂₂-2-hydroxydecyl ethers.

In particular, the end-capped poly(oxyalkylated) non-ionic surfactants of formula R¹O[CH₂CH₂O]ₓ[CH₂CH(R³)O]_{y}CH₂CH(OH)R² are preferred, in which R¹ and R² represent, independently of one another, a linear or branched, saturated or mono- or polyunsaturated hydrocarbon functional group having 2 to 26 carbon atoms, R³ is selected, independently of one another, from —CH₃, —CH₂CH₃, —CH₂CH₂—CH₃, —CH(CH₃)₂, but preferably represents —CH₃, and x and y represent, independently of one another, values of between 1 and 32, with non-ionic surfactants where R³═—CH₃ and having values for x of from 15 to 32 and for y of 0.5 and 1.5 being very particularly preferred.

Further non-ionic surfactants that can preferably be used are the end-capped poly(oxyalkylated) non-ionic surfactants of the formula R¹O[CH₂CH(R³)O]ₓ[CH₂]ₖCH(OH)[CH₂]ⱼOR², in which R¹ and R² represent linear or branched, saturated or unsaturated, aliphatic or aromatic hydrocarbon functional groups having 1 to 30 carbon atoms, R³ represents H or a methyl, ethyl, n-propyl, iso-propyl, n-butyl, 2-butyl or 2-methyl-2-butyl functional group, x represents values between 1 and 30, and k and j represent values between 1 and 12, preferably between 1 and 5. If the value x is >2, each R³ in the above formula R¹O[CH₂CH(R³)O]ₓ[CH₂]ₖCH(OH)[CH₂]ⱼOR² can be different. R¹ and R² are preferably linear or branched, saturated or unsaturated, aliphatic or aromatic hydrocarbon functional groups having 6 to 22 carbon atoms, with functional groups having 8 to 18 C atoms being particularly preferred. For the functional group R³, H, —CH₃ or —CH₂CH₃ are particularly preferred. Particularly preferred values for x are in the range of from 1 to 20, in particular from 6 to 15.

As described above, each R³ in the above formula can be different if x>2. In this way, the alkylene oxide unit in square brackets can be varied. For example, if x represents 3, the functional group R3 can be selected in order to form ethylene oxide (R³═H) or propylene oxide (R³═CH₃) units, which can be joined together in any sequence, for example (EO)(PO)(EO), (EO)(EO)(PO), (EO)(EO)(EO), (PO)(EO)(PO), (PO)(PO)(EO) and (PO)(PO)(PO). The value 3 for x has been selected here by way of example and can by all means be greater, in which case the range of variation increases as the values for x increase and includes a large number of (EO) groups combined with a small number of (PO) groups, for example, or vice versa.

Particularly preferred end-capped poly(oxyalkylated) alcohols of the above formula have values of k=1 and j=1, and therefore the previous formula is simplified to R¹O[CH₂CH(R³)O]ₓCH₂CH(OH)CH₂OR². In the formula mentioned last, R¹, R² and R³ are as defined above and x represents numbers from 1 to 30, preferably from 1 to 20, and in particular from 6 to 18. Surfactants in which the functional groups R¹ and R² have 9 to 14 C atoms, R³ represents H, and x assumes values from 6 to 15 are particularly preferred. Finally, the non-ionic surfactants of general formula R¹—CH(OH)CH₂O-(AO)_{w}—R² have been found to be particularly effective, in which
R¹ represents a straight-chain or branched, saturated or mono- or polyunsaturated C₆₋₂₄ alkyl or alkenyl functional group;
R² represents a linear or branched hydrocarbon functional group having 2 to 26 carbon atoms;
A represents a functional group from the group CH₂CH₂, CH₂CH₂CH₂, CH₂CH(CH₃), preferably represents CH₂CH₂, and
w represents values of between 1 and 120, preferably 10 to 80, in particular 20 to 40.

The group of these non-ionic surfactants includes, for example, the C₄₋₂₂ fatty alcohol-(EO)₁₀₋₈₀-2-hydroxyalkyl ethers, in particular including the C₈₋₁₂ fatty alcohol-(EO)₂₂-2-hydroxydecyl ethers and the C₄₋₂₂ fatty alcohol-(EO)₄₀₋₈₀-2-hydroxyalkyl ethers.

Preferably, the at least one solid and/or the at least one gel phase contains at least one non-ionic surfactant, preferably a non-ionic surfactant from the group of the hydroxy mixed ethers, with the proportion by weight of the non-ionic surfactant with respect to the total weight of the gel phase being preferably 0.5 wt. % to 30 wt. %, more preferably 5 wt. % to 25 wt. %, and in particular 10 wt. % to 20 wt. %.

In another preferred embodiment, the non-ionic surfactant of the solid and/or gel phase is selected from non-ionic surfactants of general formula R¹—O(CH₂CH₂O)ₓCR³R⁴(OCH₂CH₂)_{y}O—R², in which R¹ und R², independently of one another, represent an alkyl functional group or alkenyl functional group having 4 to 22 carbon atoms; R³ und R⁴ represent, independently of one another, H or an alkyl functional group of alkenyl functional group having 1 to 18 carbon atoms, and x and y represent, independently of one another, values between 1 and 40.

In particular, compounds of general formula R¹—O(CH₂CH₂O)ₓCR³R⁴(OCH₂CH₂)_{y}O—R² are preferred, in which R³ and R⁴ represent H and the indices x and y, independently of one another, assume values from 1 to 40, preferably from 1 to 15.

In particular, compounds of general formula R¹—O(CH₂CH₂O)ₓCR³R⁴(OCH₂CH₂)_{y}O—R² are particularly preferred, in which the functional groups R¹ and R², independently of one another, represent saturated alkyl functional groups having 4 to 14 carbon atoms and the indices x and y, independently of one another, assume values from 1 to 15 and in particular from 1 to 12.

In addition, such compounds of general formula R¹—O(CH₂CH₂O)ₓCR³R⁴(OCH₂CH₂)_{y}O—R² are preferred in which one of the functional groups R¹ and R² is branched.

Most particularly preferred are compounds of general formula R¹—O(CH₂CH₂O)ₓCR³R⁴(OCH₂CH₂)_{y}O—R², in which the indices x and y, independently of one another, assume values from 8 to 12.

The indicated C chain lengths and degrees of ethoxylation or degrees of alkoxylation of the non-ionic surfactants represent statistical averages that can be an integer or a fraction for a given product. Owing to the manufacturing methods, commercial products of the above-mentioned formulas generally do not consist of an individual representative, but of mixtures, for which reason average values and, resulting from those, fractional numbers can arise both for the C chain lengths and for the degrees of ethoxylation and degrees of alkoxylation.

Naturally, the above-mentioned non-ionic surfactants can be used not only as individual substances but also as surfactant mixtures of two, three, four, or more surfactants.

In the at least one solid phase, the non-ionic surfactants are particularly preferred which have a melting point above room temperature. Non-ionic surfactant(s) having a melting point above 20° C., preferably above 25° C., particularly preferably between 25 and 60° C., and particularly between 26.6 and 43.3° C., is/are particularly preferred.

Suitable non-ionic surfactants having melting or softening points in the above-mentioned temperature range include low-foaming non-ionic surfactants, for example, which can be solid or highly viscous at room temperature. If non-ionic surfactants are used which are highly viscous at room temperature, then it is preferred that they have a viscosity above 20 Pa·s, preferably above 35 Pa·s, and particularly above 40 Pa·s. Non-ionic surfactants that have a wax-like consistency at room temperature are also preferred.

The non-ionic surfactant that is solid at room temperature preferably has propylene oxide (PO) units in the molecule. Preferably, such PO units constitute up to 25 wt. %, particularly preferably up to 20 wt. %, and in particular up to 15 wt. % of the total molar mass of the non-ionic surfactant. Particularly preferred non-ionic surfactants are ethoxylated monohydroxy alkanols or alkyl phenols that additionally have polyoxyethylene-polyoxypropylene block copolymer units. The alcohol or alkyl phenol fraction of such non-ionic surfactant molecules preferably constitutes greater than 30 wt. %, particularly preferably greater than 50 wt. %, and in particular greater than 70 wt. % of the total molar mass of such non-ionic surfactants. Preferred agents are characterized in that they contain ethoxylated and propoxylated non-ionic surfactants in which the propylene oxide units in the molecule constitute up to 25 wt. %, preferably up to 20 wt. %, and particularly up to 15 wt. % of the total molar mass of the non-ionic surfactant.

Additional particularly preferred non-ionic surfactants to be used in the solid phase having melting points above room temperature contain 40 to 70% of a polyoxypropylene/polyoxyethylene/polyoxypropylene block polymer blend that contains 75 wt. % of a reverse block copolymer of polyoxyethylene and polyoxypropylene with 17 mol of ethylene oxide and 44 mol of propylene oxide and 25 wt. % of a block copolymer of polyoxyethylene and polyoxypropylene, initiated with trimethylolpropane and containing 24 mol of ethylene oxide and 99 mol of propylene oxide per mol of trimethylolpropane.

In one preferred embodiment, the proportion by weight of the non-ionic surfactant with respect to the total weight of the solid phase is from 0.1 to 20 wt. %, particularly preferably from 0.5 to 15 wt. %, and in particular from 2.5 to 10 wt. %.

All anionic surface-active substances are suitable for use as anionic surfactants in dishwasher detergents. These are characterized by a water-solubilizing, anionic group such as a carboxylate, sulfate or sulfonate group and a lipophilic alkyl group having approximately 8 to 30 carbon atoms. In addition, glycol or polyglycol ether groups, ester, ether and amide groups as well as hydroxyl groups can be contained in the molecule. Suitable anionic surfactants are preferably present in the form of sodium, potassium and ammonium salts as well as mono-, di- and trialkanolammonium salts having 2 to 4 carbon atoms in the alkanol group, but zinc, manganese(II), magnesium, calcium or mixtures thereof may also serve as counterions. Preferred anionic surfactants are alkyl sulfates, alkyl polyglycol ether sulfates, and ether carboxylic acids having 10 to 18 C atoms in the alkyl group and up to 12 glycol ether groups in the molecule.

Instead of the above-mentioned surfactants or in conjunction therewith, cationic and/or amphoteric surfactants such as betaines or quaternary ammonium compounds can also be used. It is preferred, however, that no cationic and/or amphoteric surfactants be used.

Surfactants influence the opacity of the gel phase. In another embodiment, the gel phase is therefore free from non-ionic surfactants, in particular free from surfactants. “Free from” means that the gel phase contains less than 1.0 wt. % and in particular less than 0.1 wt. %, preferably no surfactant or no non-ionic surfactant. Preferred cleaning agents are also characterized in that they contain, in the at least one solid and/or the at least one gel phase, in particular in the solid phase, less than 1.0 wt. % and in particular less than 0.1 wt. %, and preferably no anionic surfactant.

According to a particularly preferred embodiment, the cleaning agents are characterized in that the at least one gel phase comprises less than 1 wt. %, in particular less than 0.5 wt. %, in particular less than 0.1 wt. %, anionic surfactant, in each case based on the total weight of the gel phase.

The at least one gel phase is preferably substantially free of anionic surfactants. Substantially free means that the at least one gel phase comprises less than 0.05 wt. % anionic surfactant, in each case based on the total weight of the gel phase.

It has been found in this context that the presence of 1 wt. % anionic surfactant in the at least one gel phase leads to poorer foaming behavior and poorer rinsing behavior of the total composition. Furthermore, higher amounts of anionic surfactants adversely affect the curing. According to a particularly preferred embodiment, the gel phase contains less than 1 wt. %, preferably less than 0.5 wt. %, in particular less than 0.05 wt. %, fatty acid salts or soaps.

According to another embodiment, the at least one gel phase may contain sugar. Sugars include sugar alcohols, monosaccharides, disaccharides, and oligosaccharides. In a preferred embodiment, the at least one gel phase comprises at least one sugar alcohol other than glycerol, preferably at least one monosaccharide or disaccharide sugar alcohol. In particular, mannitol, isomalt, lactitol, sorbitol, threitol, erythritol, arabitol, and xylitol are preferred. Particularly preferred monosaccharide sugar alcohols are pentitols and/or hexitols. Xylitol and/or sorbitol are very particularly preferred.

In another embodiment, the gel phase can comprise disaccharides, in particular sucrose. The sucrose fraction is 0 wt. % to 30 wt. %, in particular 5 wt. % to 25 wt. %, particularly preferably 10 wt. % to 20 wt. %, with respect to the weight of the gel phase. In greater quantities, the sugar does not dissolve completely in the gel phase and results in the clouding thereof. By using sugar, in particular in a proportion of from 10 wt. % to 15 wt. %, the development of moisture is reduced and the adhesion to the at least one solid phase is thus improved.

The use of builder substances (builders) such as silicates, aluminum silicates (in particular zeolites), salts of organic di- and polycarboxylic acids, as well as mixtures of these substances, preferably water-soluble builder substances, can be advantageous.

In an embodiment that is particularly preferred, the use of phosphates (including polyphosphates) is omitted either largely or completely. In this embodiment, the agent preferably contains less than 5 wt. %, particularly preferably less than 3 wt. %, in particular less than 1 wt. %, phosphate(s). Particularly preferably, the agent in this embodiment is completely phosphate-free, i.e., the agents contain less than 0.1 wt. % phosphate(s).

The builders include in particular carbonates, citrates, phosphonates (if permissible due to regulations), organic builders, and silicates. The proportion by weight of the total builders with respect to the total weight of agents is preferably 15 to 80 wt. % and in particular 20 to 70 wt. %.

Some examples of organic builders that are suitable are the polycarboxylic acids (polycarboxylates) that can be used in the form of their sodium salts, with polycarboxylic acids being understood as being those carboxylic acids that carry more than one, in particular two to eight, acid functions, preferably two to six, in particular two, three, four, or five acid functions in the entire molecule. As polycarboxylic acids, dicarboxylic acids, tricarboxylic acids, tetracarboxylic acids, and pentacarboxylic acids, in particular di-, tri-, and tetracarboxylic acids, are thus preferred. The polycarboxylic acids can also carry additional functional groups such as hydroxyl or amino groups, for example. For example, these include citric acid, adipic acid, succinic acid, glutaric acid, malic acid, tartaric acid, maleic acid, fumaric acid, sugar acids (preferably aldaric acids, for example galactaric acid and glucaric acid), aminocarboxylic acids, in particular aminodicarboxylic acids, aminotricarboxylic acids, aminotetracarboxylic acids such as, for example, nitrilotriacetic acid (NTA), glutamic-N,N-diacetic acid (also called N,N-bis(carboxymethyl)-L-glutamic acid or GLDA), methyl glycine diacetic acid (MGDA) and derivatives thereof and mixtures thereof. Preferred salts are the salts of the polycarboxylic acids such as citric acid, adipic acid, succinic acid, glutaric acid, tartaric acid, GLDA, MGDA, and mixtures thereof.

Other substances that are suitable as organic builders are polymeric polycarboxylates (organic polymers with a plurality of (in particular greater than ten) carboxylate functions in the macromolecule), polyaspartates, polyacetals, and dextrins.

Besides their builder effect, the free acids also typically have the quality of an acidification component. Particularly noteworthy here are citric acid, succinic acid, glutaric acid, adipic acid, gluconic acid, and any mixtures thereof.

Particularly preferred cleaning agents, in particular dishwasher detergents, preferably automatic dishwasher detergents, contain one or more salts of citric acid, i.e. citrates, as one of their essential builders. These are preferably contained in a proportion of from 2 to 40 wt. %, in particular from 5 to 30 wt. %, particularly from 7 to 28 wt. %, particularly preferably from 10 to 25 wt. %, very particularly preferably from 15 to 20 wt. %, in each case based on the total weight of the agent.

It is also particularly preferred to use carbonate(s) and/or hydrogen carbonate(s), preferably alkali carbonate(s), particularly preferably sodium carbonate (soda), in quantities of from 2 to 50 wt. %, preferably from 4 to 40 wt. %, and in particular from 10 to 30 wt. %, very particularly preferably from 10 to 24 wt. %, in each case based on the weight of the agent.

Particularly preferred cleaning agents, in particular dishwasher detergents, preferably automatic dishwasher detergents, are characterized in that they contain at least two builders from the group of silicates, phosphonates (if permissible due to regulations), carbonates, aminocarboxylic acids, and citrates, with the proportion by weight of these builders based on the total weight of the cleaning agent being preferably 5 to 70 wt. %, more preferably 15 to 60 wt. %, and in particular 20 to 50 wt. %. The combination of two or more builders from the above-mentioned group has proven advantageous for the cleaning and rinsing performance of cleaning agents, in particular dishwasher detergents, preferably automatic dishwasher detergents. Beyond the builders mentioned here, one or more other builders can be additionally contained.

Preferred cleaning agents, in particular dishwasher detergents, preferably automatic dishwasher detergents, are characterized by a builder combination of citrate and carbonate and/or hydrogen carbonate. In one embodiment that is very particularly preferred, a mixture of carbonate and citrate is used in which the amount of carbonate is preferably from 5 to 40 wt. %, in particular from 10 to 35 wt. %, very particularly preferably from 15 to 30 wt. %, and the amount of citrate is preferably from 5 to 35 wt. %, in particular from 10 to 25 wt. %, very particularly preferably from 15 to 20 wt. %, in each case based on the total amount of the cleaning agent, with the total amount of these two builders preferably being from 20 to 65 wt. %, in particular from 25 to 60 wt. %, preferably from 30 to 50 wt. %. Moreover, one or more other builders can be additionally contained.

The cleaning agents, in particular dishwasher detergents, preferably automatic dishwasher detergents, can contain phosphonates (if permissible due to regulations) in particular as an additional builder, if permissible due to regulations. A hydroxy alkane and/or amino alkane phosphonate is preferably used as a phosphonate compound. Among the hydroxy alkane phosphonates, 1-hydroxyethane-1,1-diphosphonate (HEDP) has particular significance. Possible preferable aminoalkane phosphonates include ethylenediamine tetramethylene phosphonate (EDTMP), diethylentriamine pentamethylene phosphonate (DTPMP) and the higher homologs thereof. Phosphonates are preferably contained in agents (if permissible due to regulations) in amounts of from 0.1 to 10 wt. %, in particular in amounts of from 0.5 to 8 wt. %, very especially preferably from 2.5 to 7.5 wt. %, in each case based on the total weight of the agent.

The combined use of citrate, (hydrogen) carbonate, and phosphonate (if permissible due to regulations) is particularly preferred. These can be used in the above-mentioned amounts. In particular, quantities of 10 to 25 wt. % citrate, 10 to 30 wt. % carbonate (or hydrogen carbonate), and 2.5 to 7.5 wt. % phosphonate are used in this combination, in each case based on the total weight of the agent.

Additional particularly preferred cleaning agents, in particular dishwasher detergents, preferably automatic dishwasher detergents, are characterized in that, in addition to citrate and (hydrogen) carbonate and, optionally, phosphonate, they contain at least one additional phosphorus-free builder. In particular, it is selected from aminocarboxylic acids, with the additional phosphorous-free builder preferably being selected from methyl glycine diacetic acid (MGDA), glutamic acid diacetate (GLDA), aspartic acid diacetate (ASDA), hydroxyethyliminodiacetate (HEIDA), iminodisuccinate (IDS), and ethylenediamine disuccinate (EDDS), particularly preferably from MGDA or GLDA. An example of a particularly preferred combination is citrate, (hydrogen) carbonate, and MGDA as well as, optionally, phosphonate (if permissible due to regulations).

The proportion by weight of the additional phosphorous-free builder, in particular of the MGDA and/or GLDA, is preferably from 0 to 40 wt. %, in particular from 5 to 30 wt. %, more particularly from 7 to 25 wt. %. The use of MGDA or GLDA, in particular MGDA, as granulate is particularly preferred. Advantageous in this regard are MGDA granulates that contain as little water as possible and/or have a lower hygroscopicity (water absorption at 25° C., normal pressure) than non-granulated powders. The combination of at least three, in particular at least four, builders from the above-mentioned group has proven advantageous for the cleaning and rinsing performance of cleaning agents, in particular dishwasher detergents, preferably automatic dishwasher detergents. Besides those, additional builders can also be contained.

Polymeric polycarboxylates are also suitable as organic builders. These are, for example, the alkali metal salts of polyacrylic acid or polymethacrylic acid, for example those having a relative molecular mass of from 500 to 70,000 g/mol. Suitable polymers are in particular polyacrylates which preferably have a molecular mass of from 1,000 to 20,000 g/mol. Due to their superior solubility, the short-chain polyacrylates, which have molar masses of from 1,100 to 10,000 g/mol, and particularly preferably from 1,200 to 5,000 g/mol, can be preferred from this group.

The content of (homo)polymeric polycarboxylates in the cleaning agents, in particular dishwasher detergents, preferably automatic dishwasher detergents, is preferably 0.5 to 20 wt. %, preferably 2 to 15 wt. %, and in particular 4 to 10 wt. %.

Cleaning agents, in particular dishwasher detergents, preferably automatic dishwasher detergents, can also contain, as a builder, crystalline layered silicates of general formula NaMSiₓO₂ₓ₊₁·y H₂O, where M represents sodium or hydrogen, x is a number from 1.9 to 22, preferably from 1.9 to 4, with 2, 3, or 4 being particularly preferred values for x, and y represents a number from 0 to 33, preferably from 0 to 20. It is also possible to use amorphous sodium silicates with a Na₂O:SiO₂ modulus of from 1:2 to 1:3.3, preferably from 1:2 to 1:2.8, and in particular from 1:2 to 1:2.6 which preferably exhibit retarded dissolution and secondary washing properties.

In addition to the aforementioned builders, the cleaning agents can also contain alkali metal hydroxides. These alkali carriers are preferably used in the cleaning agents and in particular in the at least one gel phase only in small quantities, preferably in quantities below 10 wt. %, preferably below 6 wt. %, more preferably below 5 wt. %, particularly preferably between 0.1 and 5 wt. %, and in particular between 0.5 and 5 wt. %, in each case based on the total weight of the cleaning agent. Alternative cleaning agents are free of alkali metal hydroxides.

Besides the components cited above, the at least one solid and/or the at least one gel phase of the cleaning agent can contain additional ingredients. These include, for example, anionic, cationic and/or amphoteric surfactants, bleaching agents, bleach activators, bleach catalysts, other solvents, thickeners, sequestering agents, electrolytes, corrosion inhibitors, in particular silver protecting agents, glass corrosion inhibitors, suds suppressors, dyes, fragrances (in particular in the at least one solid phase), additives for improving the flow and drying behavior, for adjusting the viscosity, for stabilization, UV stabilizers, preservatives, antimicrobial active ingredients (disinfectants), pH adjusters in quantities of usually not more than 5 wt. %.

Agents preferably contain at least one alkanolamine as an additional solvent. The alkanolamine is preferably selected from the group consisting of mono-, di-, triethanol- and propanolamine and mixtures thereof. The alkanolamine is preferably contained in agents in a quantity of from 0.5 to 10 wt. %, in particular in a quantity of from 1 to 6 wt. %. In preferred cleaning agents, the at least one gel phase is substantially free of alkanolamine, i.e., the at least one gel phase contains less than 1 wt. %, in particular less than 0.5 wt. %, preferably less than 0.1 wt. %, particularly preferably less than 0.05 wt. %, of alkanolamine, and the alkanolamine is only contained in the at least one solid phase.

According to another preferred embodiment, the at least one gel phase of the cleaning agent, in particular dishwasher detergent, preferably automatic dishwasher detergent, contains at least one water-soluble zinc salt, preferably zinc chloride, zinc sulfate and/or zinc acetate, particularly preferably zinc acetate, for example zinc acetate anhydrate, preferably in a quantity of from 0.05 to 3 wt. %, in particular of from 0.1 to 2.4 wt. %, particularly preferably of from 0.2 to 1.0 wt. %, based on the total weight of the gel phase.

For a good incorporation ability of the zinc salts, in particular of zinc sulfate and/or zinc acetate, in particular of zinc acetate (e.g., in the water-free form of the salt) in low-water gel phases having carboxylate and/or sulfonic acid group-containing polymers, it is particularly preferred for the quantity of zinc salt in the water-free gel phase to be selected to be from 0.2 to 1.0 wt. %, for example 0.5 wt. %.

In addition to the above-mentioned zinc salts, polyethyleneimines or polyvinylamines such as those which are available under the name Lupasol® (BASF) are preferably used as glass corrosion inhibitors in a quantity of from 0 to 5 wt. %, in particular from 0.01 to 2 wt. %.

Polymers that are suitable as additives are in particular maleic acid acrylic acid copolymer Na salt (for example, Sokalan® CP 5 by BASF, Ludwigshafen (Germany)), modified polyacrylic acid Na salt (for example, Sokalan® CP 10 by BASF, Ludwigshafen (Germany)), modified polycarboxylate Na salt (for example, Sokalan® HP 25 by BASF, Ludwigshafen (Germany)), polyalkylene oxide, modified heptamethyltrisiloxane (for example, Silwet® L-77 by BASF, Ludwigshafen (Germany)), polyalkylene oxide, modified heptamethyltrisiloxane (for example, Silwet® L-7608 by BASF, Ludwigshafen (Germany)), as well as polyethersiloxane (copolymers of polymethyl siloxanes with ethylene oxide/propylene oxide segments (polyether blocks)), preferably water-soluble, linear polyether siloxanes with terminal polyether blocks, such as Tegopren® 5840, Tegopren® 5843, Tegopren® 5847, Tegopren® 5851, Tegopren® 5863, or Tegopren® 5878 by Evonik, Essen (Germany). Builders suitable as additives are in particular polyaspartic acid Na salt, ethylene diamine triacetate cocoalkyl acetamide (for example Rewopol® CHT 12 from Evonik, Essen (Germany)), methylglycine tri-Na diacetic acid salt and acetophosphonic acid. In the case of Tegopren® 5843 and Tegopren® 5863, mixtures with surface-active or polymeric additives exhibit synergies. However, the use of Tegopren types 5843 and 5863 on hard surfaces made of glass, in particular glass dishes, is less preferred, since these silicone surfactants can adhere to glass. In a particular embodiment, the above-mentioned additives are omitted.

A preferred cleaning agent, in particular an automatic dishwasher detergent, preferably also comprises a bleaching agent, in particular an oxygen bleaching agent, as well as, optionally, a bleach activator and/or bleach catalyst. Insofar as they are present, they are contained exclusively in the at least one solid phase.

As a preferred bleaching agent, cleaning agents contain an oxygen bleaching agent from the group of sodium percarbonate, sodium perborate tetrahydrate, and sodium perborate monohydrate. Further examples of bleaching agents which may be used are peroxypyrophosphates, citrate perhydrates as well as H₂O₂-yielding peracid salts or peracids, such as perbenzoates, peroxophthalates, diperazelaic acid, phthaloiminoperacid or diperdodecane diacid. Moreover, bleaching agents from the group of the organic bleaching agents can also be used. Typical organic bleaching agents are the diacyl peroxides, such as dibenzoyl peroxide. Other typical organic bleaching agents are the peroxy acids, with the alkylperoxy acids and the arylperoxy acids meriting special mention as examples. Due to its good bleaching performance, sodium percarbonate is particularly preferred. One particularly preferred oxygen bleaching agent is sodium percarbonate.

Compounds which, under perhydrolysis conditions, result in aliphatic peroxocarboxylic acids having preferably 1 to 10 C atoms, in particular 2 to 4 C atoms, and/or optionally substituted perbenzoic acid, may be used as bleach activators. Substances that carry the O- and/or N-acyl groups of the stated number of C atoms and/or optionally substituted benzoyl groups are suitable. Multiply acylated alkylene diamines are preferred, with tetraacetylethyl ethylenediamine (TAED) having proven to be particularly suitable.

The bleach catalysts are bleach-boosting transition metal salts or transition metal complexes such as, for example, Mn-, Fe-, Co-, Ru-, or Mo-salene complexes or -carbonyl complexes. Mn-, Fe-, Co-, Ru-, Mo-, Ti-, V-, and Cu complexes with N-containing tripod ligands as well as Co-, Fe- Cu-, and Ru-ammine complexes can also be used as bleach catalysts. Complexes of manganese in oxidation stage II, III, IV, or IV are particularly preferably used which preferably contain one or more macrocyclic ligands with the donor functions N, NR, PR, O and/or S. Preferably, ligands are used which have nitrogen donor functions. It is particularly preferred to use bleach catalyst(s) in the agents which contains or contain, as macromolecular ligands, 1,4,7-trimethyl-1,4,7-triazacyclononane (Me-TACN), 1,4,7-triazacyclononane (TACN), 1,5,9-trimethyl-1,5,9-triazacyclododecane (Me-TACD), 2-methyl-1-1,4,7-trimethyl-1,4,7-triazacyclononane (Me/Me-TACN), and/or 2-methyl-1,4,7-triazacyclononane (Me/TACN). Suitable manganese complexes are, for example, [Mn^{III}₂(μ-O)₁(μ-OAC)₂(TACN)₂](CIO₄)₂, [Mn^{III}Mn^{lv}(μ-O)₂(μ-O Ac)ₜ(TACN)₂](BPh₄)₂, [Mn^{lv}₄(μ-O)₆(TACN)₄](CIO₄)₄, [Mn^{III}₂(μ-O)₁(μ-OAc)₂(Me-TACN)₂](CIO₄)₂, [Mn^{III}Mn^{lv}(μ-O)₁(μ-OAc)₂(Me-TACN)₂](CIO₄)₃, [Mn^{lv}₂(μ-O)₃(Me-TACN)₂](PF₆)₂ and [Mn^{lv}₂(μ-O)₃(Me/Me-TACN)₂](PF₆)₂ (where OAc═OC(O)CH₃).

The cleaning agent preferably comprises at least one solid phase and at least one gel phase. The cleaning agent can thus have one, two, three, or more different solid phases; it may also have one, two, three or more different gel phases. The cleaning agent preferably comprises a solid phase and a gel phase. The cleaning agent particularly preferably comprises two solid phases and a gel phase. It preferably comprises two solid phases and two gel phases. An embodiment is also preferred in which the cleaning agent comprises three solid phases and one or two gel phases.

In this case, the weight ratio of the entirety of the at least one solid phase to the entirety of the at least one gel phase is usually 40:1 to 2:1, in particular 20:1 to 4:1, preferably 14:1 to 6:1, for example 12:1 to 8:1. The total weight of all phases in a cleaning agent portion can be between 8 and 30 g, in particular 10 to 25 g, preferably 12 to 21 g, for example 13 to 17 g per cleaning agent portion. This weight ratio provides a good concentration of the respective ingredients of the solid and the gel phase in a cleaning process.

According to a preferred embodiment, the protease is contained in a total amount of from 0.001 to 1,000 mg, more preferably 0.1 to 600 mg and even more preferably 1.0 to 400 mg of enzyme protein (based on active enzyme protein) in the cleaning agent portion.

The at least one solid phase and the at least one gel phase are adjacent to one another over all or part of their surfaces. It is preferred in this regard that the two phases be immediately adjacent. If the at least one solid phase and the at least one gel phase are directly adjacent to each other over all or part of their surfaces, stability is important in addition to the shortest possible solidification time of the at least one gel phase. Here, stability means that components contained in the gel phase do not cross over into the at least one solid phase, but rather the at least one solid phase and the gel phase remain visually separate from one another even after a long period of storage and do not interact with each other, for example by the diffusion of liquid components from one phase to the other or the reaction of components of one phase with those in the other phase.

The present application also relates to a method for cleaning hard surfaces, in particular dishes, in which the surface is worked in an inherently known manner using a cleaning agent. In particular, the surface is brought into contact with the cleaning agent. The cleaning is performed in particular using a cleaning machine, preferably a dishwasher.

The cleaning agent, as described above, may be used for cleaning hard surfaces, in particular dishes, in particular in automatic dishwashers. That which has been specifically disclosed above in relation to the cleaning agents also applies to the use and the method.

A particularly preferred embodiment relates to a cleaning agent which contains at least one enzyme, preferably an enzyme granulate, polyethylene glycols having an average molecular weight of 200 to 600 g/mol, glycerol, PVOH and at least one C₃ to C_{S} alkanediol. Preference is given to a single cleaning agent portion in a water-soluble wrapping which contains at least one enzyme, preferably an enzyme granulate, polyethylene glycols having an average molar mass of 200 to 600 g/mol, glycerol, PVOH and at least one C₃ to C₅ alkanediol.

Insofar as it is stated in the present application that the cleaning agent comprises something overall or in the at least one solid phase or in the at least one gel phase, this shall also be regarded as disclosing the fact that cleaning agents, or the relevant phase, can consist thereof. In the following practical example, the cleaning agent is described in a non-limiting manner.

### EMBODIMENTS

Cleaning agents were prepared that comprise a solid phase and a gel phase. Different geometries were produced in the process. In addition, cleaning agents were prepared that comprise two solid phases and a gel phase. Cleaning agents were also prepared that comprise a solid phase and three, four and five gel phases (having the same or different composition). The following quantities refer to wt. % of active substance based on the total weight of the relevant phase (unless indicated otherwise).

TABLE 1
The solid granular mixtures of a solid composition,
in particular powdery and free-flowing phases,
had the following preferred composition:

| | wt. % |
|---|---|
| Citrate, Na salt | 15-20 |
| Phosphonate (esp. HEDP) If permitted by regulation | 0-7.5 (2.5-7.5) |
| MGDA, Na salt | 0-25 |
| Disilicate, Na salt | 5-35 |
| Soda | 10-25 |
| Silver protection (especially cysteine) | 0.0-1.0 |
| Percarbonate, Na salt | 10-15 |
| Bleach catalyst (preferably Mn-based) | 0.02-0.5 |
| Bleach activator (esp. TAED) | 1-3 |
| Non-ionic surfactant(s), e.g., fatty alcohol alkoxylate, | 2.5-10 |
| preferably 20-40 EO, optionally end-capped | |
| Polycarboxylate | 4-10 |
| Cationic copolymer | 0-0.75 |
| Disintegrant - (esp. Crosslinked PVP) | 0-1.5 |
| Protease preparation (tq) | 0-5 |
| Amylase preparation (tq) | 0-3 |
| Perfume | 0.05-0.25 |
| Dye solution | 0.0-1 |
| Zn salt (e.g. acetate) | 0.1-0.3 |
| Sodium sulfate | 0.0-10 |
| Water | 0.0-1.5 |
| pH adjuster (e.g., citric acid) | 0-1.5 |
| Processing aids | 0-5 |

TABLE 2
The gel phases used had the following compositions (quantities
in each case based on the total amount of the gel phase):

| | wt. % |
|---|---|
| Enzyme granulate (protease), based on active protein | 0.01-7.5 |
| content | |
| Enzyme granulate (amylase), based on active protein | 0-5.0 |
| content | |
| Water-soluble zinc salt (preferably zinc acetate anhydrate) | 0.1-2.4 |
| Glycerol | 10-50 |
| Propanediol (preferably 1,3-propanediol) | 10-50 |
| Polycarboxylate copolymer having sulfonic acid-containing | 0-30 |
| groups, preferably Acusol ® 588 or Acusol ® 590 | |
| Non-ionic surfactant(s), e.g., fatty alcohol alkoxylate, | 0-40 |
| preferably 20-40 EO, optionally end-capped | |
| Polyethylene glycol avg. molar mass 200-600 (for example | 8-26 |
| PEG 400 (INCI)) | |
| PVOH | 8-22 |
| Processing aids | 0-10 |
| Dye solution | 0.0-1.5 |
| Misc., other active substances, organic solvents, perfume, | To make up |
| | to 100 |

The solid and gel phases could be combined as desired. The spatial configuration of the gel phase, which was liquid after mixing of the ingredients and dimensionally stable within a setting time of a maximum of 10 minutes, was predetermined by the spatial configuration of the solid phase and by molds that are commercially available or self-designed. A water-soluble wrapping in the form of an open pouch was produced by thermoforming a PVOH-containing film. A liquid composition was poured into said open cavity and resulted in the gel phase after curing, then solid phases in the form of a free-flowing solid were poured into a pouch comprising polyvinyl alcohol, and the open pouch was then sealed by applying a second film and sealing by heat sealing.

TABLE 3
Compositions of the gel phase in wt. %

| | G1 | G2 | G3 | G4 | G5 | G6 |
|---|---|---|---|---|---|---|
| Water-free zinc acetate | 0.5 | 0.5 | 1.0 | 1.0 | 2.0 | 2.0 |
| Polymer comprising acrylic acid- | 11 | 0 | 0 | 11 | 11 | 0 |
| containing and amidopropyl | | | | | | |
| sulfonic acid-containing | | | | | | |
| monomers, Acusol ® 588 | | | | | | |
| Polymer comprising acrylic acid- | 0 | 11 | 11 | 0 | 0 | 11 |
| containing and amidopropyl | | | | | | |
| sulfonic acid-containing | | | | | | |
| monomers, Acusol ® 590 | | | | | | |
| Glycerol | 25 | 25 | 25 | 25 | 25 | 25 |
| 1,3-propanediol | 30 | 30 | 30 | 30 | 30 | 30 |
| PEG 400 | 15 | 15 | 15 | 15 | 15 | 15 |
| PVOH (Mowiol 4-88) | 15 | 15 | 15 | 15 | 15 | 15 |
| Misc. (including processing | To | To | To | To | To | To |
| aids, pH | make | make | make | make | make | make |
| adjuster, perfume, dye) | up to | up to | up to | up to | up to | up to |
| | 100 | 100 | 100 | 100 | 100 | 100 |

Corresponding formulations have been prepared according to table 3. The gel phases were stirred at temperatures of 110° C. Shortly before dosing the gel phases, protease granulates 1 or 2 were dosed into the still-hot compositions.

The corresponding gel phases (1.5 g) were then packaged as described under Table 2 together with a solid phase according to Table 4 (17.0 g) in single portions (pouches) with a total weight of 18.5 g.

TABLE 4
Composition of the gel phase:

| | wt. % of active substance |
|---|---|
| Citrate, Na salt | 35 |
| Soda | 25 |
| Percarbonate, Na salt | 10 |
| Bleach catalyst (Mn-TACN) | 0.05 |
| Bleach activator (TAED) | 1 |
| Non-ionic surfactants | 2.5 |
| Polycarboxylate, (Acusol ® 588) | 5 |
| Amylase preparation (tq) | 1.5 |
| Sodium sulfate, dye, perfume | To make up to 100 |

Cleaning Performance:

Using the single portions prepared in this way (gel phase G1, solid phase, Table 4), the cleaning performance of the compositions was determined according to the IKW method in a Miele GSL, Eco 45° program, at 21° dH.

TABLE 5

| | | Creme | Egg | |
|---|---|---|---|---|
| | Cleaning performance | brûlée | yolk | Spaghetti |
| E1 | G1 + protease granulate 1 (30 mg/job) | 7.2 | 4.7 | 3.5 |
| E2 | G1 + protease granulate 2 (30 mg/job) | 8.4 | 8.0 | 4.5 |

Both protease granulates each contain 30 mg of active protein/job (corresponding to the active enzyme protein content per single portion, suitable for a cleaning application).

The protease granulate 1 contains a combination of protease 1a (protease according to SEQ ID NO:6 from WO2016000973) and protease 1b (Ovozyme®, ex Novozymes) in a ratio of 1.5:1.

The protease granulate 2 contains a protease according to SEQ ID NO:5 from WO2017215925.

Surprisingly, it was found that the proteases were able to withstand the remaining high temperatures of the gel preparation before it solidified (>90° C.). Furthermore, it was surprisingly found that when protease granulate 2 is used, in addition to a particularly good cleaning performance on protein-containing stains, an effect on the starch-based stain spaghetti can also be observed.

In a corresponding further experiment with the same parameters, only lower amounts of protease were used in the gel phase (see Table 6):

TABLE 6

| | | Burnt-on | |
|---|---|---|---|
| | Cleaning performance | minced meat | Egg yolk |
| E3 | G1 + protease granulate 1 (15 mg/job) | 4.2 | 3.2 |
| E4 | G1 + protease granulate 2 (15 mg/job) | 6.9 | 5.6 |

Both protease granules each contain 15 mg of active protein/job (corresponding to the content of active enzyme protein per single serving, suitable for a cleaning application).

The protease granulate 1 contains a combination of protease 1a (protease according to SEQ ID NO:6 from WO2016000973) and protease 1b (Ovozyme®, ex Novozymes) in a 1:1 ratio.

The protease granulate 2 contains a protease according to SEQ ID NO:5 from WO2017215925.

Here, too, it was surprisingly found that both proteases gave good results on protease-sensitive stains, in particular when using protease granulate 2, particularly good cleaning performance can be observed, in particular on egg yolk and burnt-on minced meat.

1. A cleaning agent comprising:
at least one gel phase comprising at least one enzyme therein.

2. The cleaning agent according to claim 1, wherein the proportion by weight of the at least one enzyme, based on the amount of active enzyme protein, in the at least one gel phase, based on the total weight of the at least one gel phase ranges from 0.0005 to 20.0 wt. %.

3. The cleaning agent according to claim 1, wherein the at least one enzyme is selected from the group of proteases amylases, or both.

4. The cleaning agent according to claim 1, wherein the at least one enzyme comprises at least one protease; wherein the at least one protease comprises:
a) an amino acid sequence having at least 70% sequence identity with the amino acid sequence given in SEQ ID NO:1 over its entire length and has an amino acid substitution at at least one of the positions corresponding to positions 212, 12, 43, 122, 127, 154, 156, 160, 211, or 222, each based on the numbering according to SEQ ID NO: 1;
b) an amino acid sequence which is at least 80% identical to the amino acid sequence specified in SEQ ID NO:2 over the entire length thereof and has at least one amino acid substitution at one positions 9, 15, 66, 212, and 239 in the numbering according to SEQ ID NO: 2;
c) an amino acid sequence which is at least 80% identical to the amino acid sequence specified in SEQ ID NO:2 over the entire length thereof and has an amino acid substitution at position 97 and an insertion of an amino acid between the amino acids at positions 97 and 98 in the numbering according to SEQ ID NO:2;
d) an amino acid sequence which is at least 80% identical to the amino acid sequence specified in SEQ ID NO:3 over the entire length thereof and has at least one amino acid substitution at one of the following positions 32, 33, 48-54, 58-62, 94-107, 116, 123-133, 150, 152-156, 158-161, 164, 169, 175-186, 197, 198, and 203-216 in the numbering according to SEQ ID NO:3;
e) an amino acid sequence having an amino acid sequence which is at least 80% identical to the amino acid sequence specified in SEQ ID NO:4 over the entire length thereof and has at least one amino acid substitution at one or more of the following positions 3, 4, 99, and 199 in the numbering according to SEQ ID NO:4;
f) and combinations thereof.

5. The cleaning agent according to claim 3, wherein the proportion by weight of the at least one protease, based on the amount of active enzyme protein of the at least one proteases with respect to the total weight of the cleaning agent, ranges from 0.005 to 5 wt. %.

6. The cleaning agent according to claim 3, wherein the proportion by weight of the at least one protease, based on the amount of active enzyme protein in the at least one gel phase based on the total weight of the at least one gel phase is ranges from 0.0005 to 20.0 wt. %.

7. The cleaning agent according to claim 3, wherein the amount of the at least one protease, based on the amount of active enzyme protein added per wash cycle ranges from 0.001 to 1,000 mg/job.

8. The cleaning agent according to claim 1, wherein the at least one enzyme comprises one or more alpha-amylases; selected from the group consisting of
a) an α-amylase having an amino acid sequence which is at least 80% identical to the amino acid sequence specified in SEQ ID NO:5 over the entire length thereof and has at least one amino acid substitution at one of the positions 172, 202, 208, 255, and 261 in the numbering according to SEQ ID NO:5;
b) an α-amylase having an amino acid sequence which is at least 60% identical to the amino acid sequence specified in SEQ ID NO:6 over the entire length thereof and has at least one amino acid substitution at one of the positions 9, 26, 30, 33, 82, 37, 106, 118, 128, 133, 149, 150, 160, 178, 182, 186, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 378, 383, 419, 421, 437, 441, 444, 445, 446, 447, 450, 458, 461, 471, 482, and 484 and/or a deletion at one of the positions 183 and 184 in the numbering according to SEQ ID NO:6;
c) an α-amylase having an amino acid sequence which is at least 90% identical to the amino acid sequence specified in SEQ ID NO:7 over the entire length thereof and has at least one substitution and/or deletion at one of the positions 93, 116, 118, 129, 133, 134, 140, 142, 146, 147, 149, 151, 152, 169, 174, 183, 184, 186, 189, 193, 195, 197, 198, 200, 203, 206, 210, 212, 213, 235, 243, 244, 260, 262, 284, 303, 304, 320, 338, 347, 359, 418, 431, 434, 439, 447, 458, 469, 476, and 477 in the numbering according to SEQ ID NO:7;
d) and combinations thereof.

9. The cleaning agent according to claim 1, further comprising a gelling agent, amount ranging from 4 to 40 wt. %, in each case based on the total weight of the gel phase.

10. The cleaning agent according to claim 1, wherein the at least one gel phase further comprises at least one organic solvent selected from the group consisting of 1,2-propanediol, 1,3-propanediol, glycerol, 1,1,1-trimethylolpropane, triethylene glycol, dipropylene glycol, polyethylene glycols, and/or mixtures thereof.

11. The cleaning agent according to claim 10, wherein the at least one organic solvent is present in the at least one gel phase in an amount ranging from 30 to 90 wt. %, based on the total weight of the at least one gel phase.

12. The cleaning agent according to claim 1, wherein the gel phase further comprises a sulfonic acid group-containing monomer.

13. The cleaning agent according to claim 1, further comprising at least one solid phase.

14. A packaging comprising a water-soluble wrapping having one or more chambers/compartments comprising the cleaning agent according to claim 1.

15. The cleaning agent according to claim 13, wherein the at least one gel phase is contained in direct contact; with the at least one solid phase.

16. (canceled)


SEQUENCE LISTING (7 sequences)

SEQ ID NO: 1 - protein, 269 aa, Bacillus gibsonii
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASMPGTSMATPHVAGVAALVKQKNPSWNATQIRNHLKNTATNLGNSSQFGSGLVNADAATR

SEQ ID NO: 2 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 3 - protein, 269 aa, Bacillus alkalophilus
AQSVPWGISRVQAPAAHNRGLTGSGVKVAVLDTGISTHPDLNIRGGASFVPGEPSTQDGNGHGTHVAGTIAALNNSIGVLGVAPNAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 4 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 5 - protein, 485 aa, Bacillus sp.
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 6 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 7 - protein, 485 aa, Bacillus sp.
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR